SEQUENCE LISTING (9 sequences)

SEQ ID NO: 1 - protein, 15 aa, Artificial Sequence
  Description of Artificial Sequence Synthesised
SLRPAPPPISGGGYR

SEQ ID NO: 2 - protein, 15 aa, Artificial Sequence
  Description of Artificial Sequence Synthesised
LHADPDLGVLCPTGC

SEQ ID NO: 3 - protein, 15 aa, Artificial Sequence
  Description of Artificial Sequence Synthesised
QLQEALLQQERPIRN

SEQ ID NO: 4 - protein, 15 aa, Artificial Sequence
  Description of Artificial Sequence Synthesised
SVDELNNNVEAVSQT

SEQ ID NO: 5 - protein, 11 aa, Artificial Sequence
  Description of Artificial Sequence Synthesised
SSSSFQYMYLL

SEQ ID NO: 6 - protein, 14 aa, Artificial Sequence
  Description of Artificial Sequence Synthesised
GHRPLDKKREEAPC

SEQ ID NO: 7 - protein, 12 aa, Artificial Sequence
  Description of Artificial Sequence Synthesised
KVERKAPDAGGC

SEQ ID NO: 8 - protein, 12 aa, Artificial Sequence
  Description of Artificial Sequence Synthesised
ARPAKAAATQKC

SEQ ID NO: 9 - protein, 16 aa, Drosophila melanogaster
RQIKIWFQNRRMKWKK

(19) United States
(12) Patent Application Publication
(10) Pub. No.: US 2004/0132969 A1
(43) Pub. Date: Jul. 8, 2004

(54) Antibodies, peptides, analogs and uses thereof

(76) Inventors: William Thomas Melvin, Aberdeen (GB); William Douglas Thompson, Inverurie (GB); Christina Maureen Stirk, Stonghaven (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/450,073

(22) PCT Filed: Dec. 12, 2001
(86) PCT No.: PCT/GB01/05505

(30) Foreign Application Priority Data
Dec. 12, 2000 (GB) 0030309.9

(51) Int. Cl. C07K 1/00; C07K 14/00; C07K 17/00

(52) U.S. Cl. 530/350

(57) ABSTRACT

Fibrin degradation products stimulate cell proliferation and angiogenesis. The present invention provides peptides, analogs and antibodies which are useful in the modulation of fibrin fragment E activities such as modulation of cell proliferation.

### FIELD OF THE INVENTION

The present invention relates broadly to compounds which act as modulators of a fibrin fragment E activity, or mimics of fibrin fragment E, and their use.

Introduction

Basic Pathology of Chronic Inflammation, Healing and Repair:

Fibrinogen is the major circulating plasma protein involved in blood clot formation. Activation of the clotting enzyme cascade, by for example injury or inflammation, results in conversion of prothrombin to thrombin which cleaves two small fibrinopeptides (A and B) from each soluble fibrinogen molecule to give fibrin monomer. Fibrin monomer comprises 6 polypeptide chains with the subunit structure (αβγ)₂. Cross linkage of monomers is the final step of the coagulation system that gives solid fibrin. Whole blood includes platelets and forms blood clots in wounds, the fibrin forming a mesh which entraps platelets and red blood cells, the clot being termed a thrombus when in abnormal arteries and veins. Inflammatory exudate is platelet free and forms fibrin alone.

Fibrin deposition and degradation is a major feature of the pathology of acute and chronic inflammation at any site in the body regardless of the underlying disease aetiology. This process is apparent at the histological level in the healing wound, the organising thrombus, the advanced therosclerotic plaque, and many other types of pathological lesions including the growing edge of some types of cancer. The fibrin mesh provides a provisional matrix for cell ingrowth, being progressively invaded in wound healing by inflammatory cells (macrophages), new small blood vessels (capillary buds), connective tissue cells (fibroblasts) and the epidermis (squamous epithelium). In the context of the large arteries subject to atherosclerosis, the endothelium of the luminal surface and the smooth muscle cells of the vascular wall invade the fibrin mesh. Secretion of plasminogen activator is the common factor that provides controlled lysis of the fibrin substratum via plasmin degradation, releasing fibrin degradation products. Fibrin degradation products are composed of combinations of two moieties termed fragments D and E. Eventually the fibrin present is replaced by new cells and matrix forming new tissue. These basic features apply to many types of human and animal disease.

Fibrin Degradation Products:

Although fibrin may be a factor common to many pathologies involving cell proliferation, it has generally been assumed that its main function was to provide an inert physical matrix to support cell movement. However there has been evidence for some time that fibrinopeptides and fibrin degradation products (FDPs) have biological activity particularly as soluble mediators of chemotaxis, the phenomenon of directional cell movement (1). It has been proposed that fibrin degradation products are a major pathological growth factor common to all sites of chronic inflammation. Using the chick chorioallantoic membrane as an in vivo test model for detection of angiogenic growth factors it has been suggested that fibrin degradation products induce cell proliferation and angiogenesis (2), have stimulatory effects on collagen synthesis (3), induce fibrogenesis, and that specifically fibrin fragment E may be the active component (4).

Fibrin deposition and lysis is believed to be relevant to a wide spectrum of human diseases including vascular restenosis, cancer, atherogenesis, rheumatoid arthritis, diabetes and renal diseases.

U.S. Pat. No. 5,981,697 describes generation of antibodies which bind fibrinogen fragments E1, E2 and E3. Bach et al (1998) J Biol Chem 273 pp30719-30728 describes an interaction of the fibrinogen β15-42 sequence with endothelial cell VE-cadherin. Lee et al (1999), Molecules and Cells 9(1) 7-13 describes the stimulation of production of IL-6 in macrophages by fragment E of fibrin and fibrinogen.

EP 0 605 797A1 (Behringwerke) discusses certain peptides related to Fibrin Fragment E.

WO 00/75175 (University Court of the University of Aberdeen) published after the earliest presently claimed priority date, discloses compounds (peptides, analogs and antibodies) which act as modulators of fibrin fragment E binding and activity. These were developed from phage combinatorial libraries.

### SUMMARY OF THE INVENTION

The present inventors have demonstrated that fibrin fragment E binds to a cell membrane component of approximately 66 kDa in size using, for example, ligand blotting of SDS-PAGE gels of cell membranes from chick fibroblasts. The presence of a specific binding site for fragment E raises the possibility that agonists and antagonists of its binding could be used to modulate its binding and thus modulate its effects. Such effects include induction of cell proliferation, angiogenesis, fibrogenesis and collagen synthesis. Such agonists and antagonists, such as those disclosed herein, may also have modulatory effects on, for example, cell stimulation per se. Such modulation may be very useful in the control of cell proliferation seen in atherosclerosis, and particularly in post angioplasty restenosis.

The production of antibodies to fibrin fragment E has been previously described, the antibodies being derived using an Fd phage combinatorial library of random epitope display to select clones binding and common to both polyclonal rat and rabbit anti E blocking antisera (32).

In the present invention, the inventors have individualised a number of compounds which modulate the induction of cell proliferation induced by fibrin degradation products and/or which per se modulate the induction of cell proliferation, and activities such as the formation of capillary-like tubules by human endothelial cells.

By contrast to the phage display approach used in WO 00/75175, the inventors have designed a number of short peptides which correspond to portions of the fibrin/ogen β chain sequence. (References to fibrin/ogen should be taken to mean fibrin and fibrinogen). The inventors began by designing a number of peptides corresponding to regions at which plasmin cleaves the fibrin/ogen molecule. It was hypothesised that these regions would be exposed and thus may represent potential active sites of the molecule. However, as described below, antibodies raised to these peptides had no effect on FDP induced stimulation. However, the inventors then designed and tested some peptides based on sequences at other sites of the molecule and antibodies raised to those peptides. Surprisingly, the inventors have shown that antibodies raised to these peptides competitively inhibit FDP induced cell proliferation. The amino acid sequences of the peptides may therefore correspond with the amino acid sequence of an active site or sites which binds to an active site of the fibrin fragment E receptor. Moreover, some of the peptides disclosed herein are demonstrated to have inherent modulatory effects on cell stimulation per se. Similarly peptides have been shown to modulate (stimulate or inhibit, in a dose dependent manner) activities such as the formation of microvascular structures by human endothelial cells.

Accordingly, the present invention provides a peptide consisting of any one of the following sequences:

| | | |
|---|---|---|
| SLRPAPPPISGGGYR | (SEQ ID NO: 1) | (WTM33) |
| LHADPDLGVLCPTGC | (SEQ ID NO: 2) | (WTM34) |
| QLQEALLQQERPIRN | (SEQ ID NO: 3) | (WTM35) |
| SVDELNNNVEAVSQT | (SEQ ID NO: 4) | (WTM36) |
| SSSSFQYMYLL | (SEQ ID NO: 5) | (WTM37) |

or a variant thereof. Variants include substitutions, insertions and deletions. Preferably, the variant retains the capability of modulating fibrin fragment E activity and/or of the ability to per se modulate (e.g. stimulate) cell proliferation or other activities such as the formation of capillary-like tubules by human endothelial cells.

Where peptides of the invention are described, it will be understood that reference is also being made to variant peptides of the invention unless the context explicitly indicates otherwise.

As described herein, fibrin fragment E activity refers to at least one of following activities: induction of cell proliferation, angiogenesis, fibrogenesis and collagen synthesis.

In another aspect of the invention there is provided a fusion peptide which comprises a first portion having the amino acid sequence of a peptide according to the invention as defined above and a second portion, attached to the N- or C-terminus of the first portion, which comprises a sequence of amino acid not naturally contiguous to the first portion. Such heterologous peptide fusions are also referred to herein as peptides of the invention.

In a further aspect, the invention provides assay methods for the identification of substances which bind to or modulate the activity of peptides of the invention, either in monomeric or oligomeric form. References to modulation of activity should be understood to include increases or decreases of activity.

In a further aspect of the invention, “analogs” of the peptides of the invention are provided. Analogs are non-peptide compounds which share fibrin fragment E activity, for example the ability to competitively inhibit binding of FDPs, for example, fibrin fragment E to the fragment E receptor, and/or the ability to stimulate cell proliferation. Analogs are a further aspect of the invention inasmuch as they are novel. Analogs may be produced by any of the techniques described herein or may be derived using, for example, combinatorial chemical libraries known in the art. Examples of such libraries are reviewed in Newton G R, Exp. Opin. Ther. Patents (1997) 7(10): 1183-1194. Where the term “chemical library” is used herein, those skilled in the art will understand its meaning accordingly.

As described below, antibodies have been raised to the peptides of the invention. The antibodies have been shown to modulate the stimulatory effect of FDPs on cell proliferation. Therefore, the invention also provides antibodies and binding fragments thereof capable of selectively binding to peptides or analogs of the invention.

In a further aspect, the invention provides a pharmaceutical composition comprising a peptide, analog or antibody according to the invention together with a pharmaceutically acceptable carrier or diluent.

Peptides, analogs or antibodies and compositions of the invention may be used for a number of purposes. They are also useful as research agents to investigate the receptor for fibrin fragment E and the activation of cell proliferation by fibrin degradation products. They may also be useful in modulating fibrin fragment E activities such as cell proliferation, and particularly in the inhibition or stimulation of angiogenesis.

Thus in a preferred aspect the peptides, analogs or antibodies of the invention may be used in a method of modulating fibrin fragment E activity, said method comprising introducing an effective amount of a peptide (or analog or antibody) of the invention. The method may be practised in vitro or in vivo. Where it is practised in vivo the invention will find use in a method of treatment of the human or animal body, particularly in methods of treating cancer or other proliferative diseases, including restenosis, e.g. caused by regrowth of vascular cells following angioplasty procedures.

The particular activities of the various peptides of the present invention in various assays are described in more detail in the Examples hereinafter. Experiments performed included: (1) A CAM cell proliferation assay using either the peptide alone or in the presence of FDP, (2) A CAM vessel counting/angiogenesis assay using the peptide alone, (3) An assay based on co-culture of HUVEC and fibroblasts, the Angiosys™ system, using the peptide alone, (4) Effect in a metastasis Lewis lung carcinoma model, and (5) Effect in a primary tumour model using Lewis lung carcinoma.

Based on the unexpected results found in these assays, the activities and utilities of the peptides can be summarised as follows.

Peptide WTM33

This gave a maximum of 50% stimulation of cell proliferation at a dose of 14 micromoles in the CAM proliferation assay. The activity dropped rapidly at high doses suggesting that high doses may be inhibitory. Peptide WTM33 was shown to reduce the FDP induced cell proliferation in the CAM model by up to 25%. Likewise, it showed stimulation at low doses of CAM vessel number in the order of 50% however at higher doses it was definitely inhibitory resulting in a 50% reduction in vessel number (p<0.05). In the Angiosys™ system it showed total inhibition of tubule formation down to concentrations of 2 micromoles/ml. Although there was no significant reduction in metastatic episodes in the tumour Metastasis Lewis Lung Carcinoma model, in the tumour Primary Lewis Lung Carcinoma model the peptide exhibited a >30% decrease in the size of the tumour at 500 microgrammes/ml.

These results show that at low doses WTM33 peptide has a mild stimulatory effect. However at high doses, and in the presence of fibrin and its degradation products, the peptide has an inhibitory effect. The inhibitory effect in the Angiosys™ model even at ‘low’ doses is likely to be a result of the nature of the model. All the other systems have a circulatory system, which results in the rapid removal of the peptide whereas the culture model is treated with the agent and remains in contact throughout the experiment. This is the equivalent of constant treatment and effectively works out to be a much higher dose than is maintained in systems in which there is a circulatory system.

Peptide WTM34

WTM34 showed inhibitory effects in the CAM assay across the range of concentrations and reaching a maximum of 60% inhibition at the highest dose tested (20 micromoles). The peptide also showed some inhibitory effect in the presence of fibrin, with possibly also a competitive effect. At low doses peptide appeared to have a stimulatory effect on vessel number, however, at 30 microgrammes/ml it resulted in a significant reduction in the number of vessels.

This again suggests that fibrin effects must be overcome before the effect is evident. The peptide in the Angiosys™ system showed inhibition of tubule formation right across the range of concentrations tried however, and it did not have a toxic effect as the number of fibroblasts and endothelial cells present remained constant. The peptide in the tumour metastasis model gave 35% reduction in the increase in weight of the lungs.

Thus peptide 34 has inhibitory effects on cell proliferation and angiogenesis, and it also reduces the metastatic potential of the Lewis lung carcinoma model.

Peptide WTM35

This peptide showed a generally stimulatory effect in the CAM assay across the range tested. The stimulatory effect of the peptide was additive to that of fibrin, up to maximum stimulation by FDP, then the activity plateaued but still continued above control level over all concentrations. The peptide also, generally, showed increased number of vessels per mm of CAM across the range of concentrations tested, only reducing to control level at very high doses. Peptide WTM35 exhibited stimulation of tubule formation at low doses and inhibition only at very high doses (which may simply reflect a generic feature of very high doses within the assay itself). It also showed an increase in the weight of lungs (approximately 20%) indicating an increase in metastatic activity in that model.

Thus it can be seen that WTM35 stimulates cell proliferation and vessel number (i.e. angiogenesis).

Peptide WTM36

Peptide WTM36 has a stimulatory effect (up to 50%) on the CAM, this appears to be a biphasic reaction reducing to control level around the 8 micromole mark but returning to significant stimulation at high doses. The effect appears to be additive to that of FDP. The peptide showed significant stimulation of tubule formation at low concentration. It also exhibited an increase in lung weight in the Lewis lung metastasis model.

These results are consistent with peptide 36 stimulating cell proliferation and angiogenesis.

Peptide WTM37

Peptide WTM37 alone had only a slight stimulatory effect on cell proliferation in the CAM. At high dose it exhibited slight inhibition of cell proliferation. In conjunction with FDP it exhibited a suppression of the FDP induced activity at 3 micromoles but did not alter it at any other concentration.

The peptide showed inhibition of tubule formation in the Angiosys™ assay but only at much higher doses than those required by the other peptides tested. Peptide 37 is thus the weakest of the peptides in the assays used herein.

Preferred Embodiments

As described above, WTM34 (and WTM33) have been shown to have an inherent inhibitory effect on cell stimulation. A preferred application for WTM33 and WTM34 would be to slow down healing to reduce scarring occurring, for example scarring associated with plastic surgery. Prevention of restenosis due to regrowth of smooth muscle cells after angioplasty may be achieved, for example, by coating stents or slow release and targeting as described in more detail hereinafter. Such peptides and their analogs may therefore find use in diseases in which uncontrolled proliferation of cells may play a part, for example, atherogenesis, rheumatoid arthritis, complications of diabetes such as retinopathies, and renal disease and psoriasis.

The inhibitory effect of particular peptides has been shown to extend to an inherent inhibitory activity on the formation of capillary-like, anastomosing, tubular structures by human endothelial cells cultured in an in vitro model of angiogenesis. Hence such peptides and their analogues may find use in diseases in which uncontrolled proliferation of new blood vessels may play a part e.g. macular degeneration, or other proliferative diseases or related treatments discussed above. More specifically, in the light of these results, the essentially inhibitory peptides WTM33 and WTM34 may be preferred for use in the treatment of cancer, especially lung cancer. As described in more detail hereinafter, the compounds may be used (for example) systemically by injection, slow release bolus or by targeting through attachment to a site directed compound (possibly to localised receptor). An alternative application if treating lung cancer patients may be direct inhalation (by analogy with therapeutics given to asthma suffers).

Particular peptides of the invention have been found to have an inherent cell proliferative activity. Of these stimulators peptides, WTM35, WTM36 (and WTM33, but only at very low concentrations) were found to have cell proliferative effects at concentrations at which FDPs no longer stimulate cell proliferation. Such peptides (and analogs or antibodies of the invention) may be useful in modulating cell proliferation per se i.e. even in the absence of fibrin degradation products.

The effects of such peptides appears to be additive to that of FDPs and so where it is practised in vivo the invention will find use in a method of treatment of the human or animal body, particularly in methods of treatment where sustained cell growth is required without interference with the effects of fibrin or fibrin degradation products per se, for example in treatments relating to wound healing or ulcers.

More specifically, peptides WTM35 and WTM36 may be preferred for use in the pro-angiogenic market, including topical application in the area of poor wound healing, and increasing effectiveness of skin grafts for burns patients etc in plastic surgery field. Other uses include assisting revascularisation of heart muscle after heart attacks, or in ischaemia due to blockage of vessels. Other uses of pro-angiogenic agents include use in limb replacement surgery.

Where activity is dose dependent, those skilled in the art will be able to apply the peptides in appropriate concentrations to achieve the desired effect.

Thus in a further preferred aspect of the invention, peptides analogs or antibodies of the invention may be used as a method of modulating cell proliferation and\or preventing angiogenesis (e.g. by inhibiting the formation of endothelial cells into tubular capillary structures) said method comprising introducing an effective amount of the peptide, analog or antibody of the invention. The method may be practised in vitro or in vivo.

### DETAILED DESCRIPTION OF THE INVENTION

“Peptides” of the invention should be understood to include variants thereof, as well as heterologous fusions of said peptides.

The term “comprising” means “including” and allows for the presence of further amino acid sequences, or other chemical moieties, at the N- and/or C-termini, provided that the peptide as a whole retains the ability to bind to a fibrin fragment E binding site and\or modulate a fibrin fragment E activity.

It is preferred that peptides of the invention (including variants of SEQ ID NOs 1-5 above and other sequences of the invention described herein), excluding any heterologous fusion sequence, are from 5 to 16, e.g. 5 to 8, 5 to 10, 8 to 16 or 8 to 11 amino acids in length.

The presence of a heterologous fusion sequence, which in itself has no activity in binding to the fibrin fragment E binding site or in cell stimulation will vary according to its intended function. Such functions include the ability to translocate across membranes, an epitope function to allow for purification or identification of the peptide, and the like. As a rough guide, the heterologous fusion sequence will be from 4 to 500, such as from 4 to 100 or 10 to 50, e.g from 10 to 30 amino acids in size.

The second peptide portion can be any sequence selected by those of skill in the art taking into account the intended purpose of the fusion. For example, the second portion may comprise a detectable tag such as a T7 tag, HA tag or a myc tag allowing identification of the peptide in a cell and/or its recovery. The second portion may also be a signal sequence directing expression of the peptide from a host cell in which the fusion is being expressed. This will be useful for the recombinant production of peptides of the invention.

The second portion may also comprise a molecular tag which influences the overall structure. A number of helix initiators which aid the formation of α-helixes which comprise short peptide sequences are known in the art.

In a preferred embodiment the second peptide portion is a membrane translocation sequence, capable of directing a peptide through the membrane of a eukaryotic cell. Examples of such peptides include the HSV-1 VP22 protein (Elliot et al, 1997), the HIV Tat protein (for example residues 1-72, 37-72 or 48-60; Fawell et al, 1994) or a sequence that is derived from the Drosophila melanogaster antennapedia protein, e.g. the 16 amino acid peptide sequence: Arg-Gln-Ile-Lys-Ile-Trp-Phe-Gln-Asn-Arg-Arg-Met-Lys-Trp-Lys-Lys.

A translocation peptide may be at the N-terminus or the C-terminus of the heterologous fusion.

Unless the context requires otherwise, reference below to peptides of the invention includes the fusion peptides described above.

Certain amino acid residues of SEQ ID NOS: 1-5 above may be substituted without significant loss of the ability of the peptide to bind to the fibrin fragment E receptor binding site and/or the ability to stimulate cell proliferation. Thus, amino acids of these peptides may be substituted to provide variant peptides which form a further aspect of the invention, within the above-described ranges.

Substitutions may include conserved substitutions, for example according to the following table, where amino acids on the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

Alternatively, any amino acid may be replaced by a small aliphatic amino acid, preferably glycine or alanine.

In addition, deletions and insertions may also be made. Insertions are preferably insertions of small aliphatic amino acids, such as glycine or alanine, although other insertions are not excluded.

Deletions may be from any part of the peptide, including either or both ends. Therefore variants also encompass fragments which could be derived by deletion of amino acids at either or both ends of SEQ ID NOS: 1 to 5.

Variants may consist of amino acid variations of from one amino acid to half of the total number of the amino acids of the peptide, for example from one to four, preferably from one to three, more preferably one or two amino acid variations. Variant peptides may also be modified in any of the ways described herein for peptides of the invention. This includes for example “reverse” C-terminal to N-terminal sequences, synthetic amino acids, modified side chains and labelling.

Where methods for the production and use of peptides of the invention are described, it will be understood that reference is also being made to variant peptides of the invention unless the context explicitly indicates otherwise.

In another aspect, a peptide of the invention may be provided in the form of molecules which contain multiple copies of the peptide (or mixtures of peptides of the invention). For example, the amino group of the side chain of lysine may be used as an attachment point for the carboxy terminus of an amino acid. Thus two amino acids may be joined to lysine via carbonyl linkages, leading to a branched structure which may in turn be branched one or more times. By way of example, four copies of a peptide of the invention may be joined to such a multiple antigen peptide (MAP), such as a MAP of the structure Pep₄-Lys₂-Lys-X, where Pep is a peptide of the invention (optionally in the form of a heterologous fusion), Lys is lysine and X is a terminal group such as β-alanine which provides for joining of the MAP core to a solid support such as a resin for synthesis of the Pep₄-MAP peptide and which may be removed from the support once synthesis is complete.

Linear multimers of peptides of the invention may also be provided.

Other multiple peptide structures may be obtained using the MAP cores described in: Lu et al, 1991, Mol Immunol, 28, 623-30; Briand et al, 1992, J Immunol Methods, 156, 255-65; Ahlborg, 1995, J Immunol Methods, 179, 269-75.

A multimer of peptides of the present invention may be fused to a translocation peptide for directing it through the membrane of a eukaryotic cell, as discussed herein. A translocation peptide may be fused to an N-terminus or a C-terminus of the multimer, or it may be incorporated at an intermediate position within the multimer.

Where multimers of the invention are provided, they may comprise different peptides of the invention or be multimers of the same peptide.

Except where specified to the contrary, the peptide sequences described herein are shown in the conventional 1-letter code and in the N-terminal to C-terminal orientation. The amino acid sequence of peptides of the invention may also be modified to include non-naturally-occurring amino acids or to increase the stability of the compound in vivo. When the compounds are produced by synthetic means, such amino acids may be introduced during production. The compound may also be modified following either synthetic or recombinant production.

Peptides of the invention may be made synthetically or recombinantly, using techniques which are widely available in the art. Synthetic production generally involves step-wise addition of individual amino acid residues to a reaction vessel in which a peptide of a desired sequence is being made.

Peptides of the invention may also be made synthetically using D-amino acids. In such cases, the amino acids will be linked in a reverse sequence in the C to N orientation. This is conventional in the art for producing such peptides.

A number of side-chain modifications for amino acids are known in the art and may be made to the side chains of peptides of the present invention. Such modifications include for example, modifications of amino groups by reductive alkylation by reaction with an aldehyde followed by reduction with NaBH₄, amidination with methylacetimidate or acylation with acetic anhydride.

The guanidino groups of arginine residues may be modified by the formation of heterocyclic condensation products with reagents such as 2,3-butanedione or glyoxal. Sulphydryl groups may be modified by methods such as carboxymethylation, tryptophan residues may be modified by oxidation or alkylation of the indole ring and the imidazole ring of histidine residues may be modified by alkylation.

The carboxy terminus and any other carboxy side chains may be blocked in the form of an ester group, e.g. a C₁₋₆alkyl ester.

The above examples of modifications to amino acids are not exhaustive. Those of skill in the art may modify amino acid side chains where desired using chemistry known per se in the art.

In another aspect, the invention provides nucleic acids encoding peptides of the invention. Such nucleic acids are free or substantially free of nucleic acid naturally contiguous with the nucleic acid encoding the peptide, for example, in the human, except possibly one or more regulatory sequences for expression.

Nucleic acids of the invention can be incorporated into a recombinant replicable vector. The vector may be used to replicate the nucleic acid in a compatible host cell. Thus in a further embodiment, the invention provides a method of making nucleic acids of the invention by introducing a nucleic acid of the invention into a replicable vector, introducing the vector into a compatible host cell, and growing the host cell under conditions which bring about replication of the vector. The vector may be recovered from the host cell. Suitable host cells are described below in connection with expression vectors.

Preferably, a nucleic acid of the invention in a vector is operably linked to a control sequence which is capable of providing for the expression of the coding sequence by the host cell, i.e. the vector is an expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell to provide for expression of a peptide of the invention. Thus, in a further aspect the invention provides a process for preparing peptides according to the invention which comprises cultivating a host cell transformed or transfected with an expression vector as described above under conditions to provide for expression by the vector of a coding sequence encoding the peptides, and recovering the expressed peptides.

The vectors may be for example, plasmid, virus or phage vectors provided with an origin of replication, optionally a promoter for the expression of the said nucleic acid and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell.

A further embodiment of the invention provides host cells transformed or transfected with the vectors for the replication and expression of nucleic acids of the invention. The cells will be chosen to be compatible with the said vector and may for example be bacterial, yeast, insect or mammalian.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters include the SV40 large T antigen promoter, retroviral LTR promoters and adenovirus promoters. All these promoters are readily available in the art.

The vector may also be adapted to be used in vivo, for example in a method of therapy. Vectors suitable for use in therapy include adenoviral vectors, retroviral vectors and alphavirus vectors. Such vectors are adapted for use in therapy by a number of modifications, for example by making the vector replication defective. Reference may be made to, for example, WO95/14091 for a description of retroviral vectors and WO95/07994 for a description of alphavirus vectors. The disclosures of both references are hereby incorporated by reference.

Vectors for use in therapy will generally be administered in the form of packed viral particles containing the vector, the particles being delivered to the site of fibrin fragment E activity, for example a tumour or other proliferating cells.

Vectors for production of peptides of the invention or for use in gene therapy include vectors which carry a mini-gene sequence of the invention.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

A peptide or analog according to the present invention may be used as an immunogen or otherwise in obtaining specific antibodies. Antibodies are useful in purification and other manipulation of peptides or analogs, diagnostic screening and therapeutic contexts. This is discussed further below.

Polyclonal antibodies which block the cell proliferative activity of fibrin fragment E can be raised by injection of the whole fragment E in rabbits. When admixed with fibrin degradation products the cell proliferative activity is abolished (17). The provision of the peptides or analogs of the invention also enables for the production of antibodies which may bind the portion of fibrin fragment E which interacts with its receptor in a specific manner. Thus the invention provides an antibody which is able to bind specifically to a peptide or analog of the invention or such portion. Such antibodies may be produced using epitopes of peptides or analogs of the invention.

Therefore, another feature of the present invention is the generation of anti-fragment E antibodies which prevent the binding of the fibrin fragment E to its receptor.

For instance, purified peptides of the invention, or a variant thereof, e.g. produced recombinantly by expression from encoding nucleic acid therefor, may be used to raise antibodies employing techniques which are standard in the art. Antibodies and peptides or analogs comprising antigen-binding fragments of antibodies may be used as discussed further below.

Methods of producing antibodies include immunising a mammal (e.g. human, mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest (which may be labelled). This is discussed below.

For instance, Western blotting techniques or immunoprecipitation may be used (Armitage et al, 1992, Nature 357: 80-82). Antibodies may be polyclonal or monoclonal.

Antibodies may be modified in a number of ways. Indeed the term “antibody” should be construed as covering any specific binding substance having a binding domain with the required specificity. Thus, this term covers antibody fragments, derivatives, functional equivalents and homologues of antibodies, including any peptide comprising an immunoglobulin binding domain, whether natural or synthetic. Chimaeric molecules comprising an immunoglobulin binding domain, or equivalent, fused to another peptide are therefore included. Cloning and expression of Chimaeric antibodies are described in EP-A-0120694 and EP-A-0125023. It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989) which consists of a VH domain; (v) isolated CDR regions; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P Holliger et al Proc. Natl. Acad. Sci. USA 90 6444-6448, 1993).

Diabodies are multimers of peptides, each peptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one peptide within the multimer with the second domain of another peptide within the multimer (WO94/13804).

As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Immunoassays for detecting antibodies are well known in the art and will generally comprise:

(a) providing a peptide comprising an epitope bindable by an antibody against said peptide;

(b) incubating a biological sample with said peptide under conditions which allow for the formation of an antibody-antigen complex; and

(c) determining whether antibody-antigen complex comprising said peptide is formed.

A peptide or analog of the invention may be labelled with a revealing label. The revealing label may be any suitable label which allows the peptide or analog to be detected. Suitable labels include radioisotopes, e.g. ¹²⁵I, enzymes, antibodies, nucleic acids and linkers such as biotin. Labelled peptides or analogs of the invention may be used in diagnostic procedures such as immunoassays in order to determine the amount of a peptide or analog of the invention in a sample. Peptides or analogs or labelled peptides or analogs of the invention may also be used in serological or cell mediated immune assays for the detection of immune reactivity to said peptides or analogs in animals and humans using standard protocols.

A peptide or analog or labelled peptide or analog of the invention or fragment thereof may also be fixed to a solid phase, for example the surface of an immunoassay well or dipstick.

Such labelled and/or immobilized peptides or analogs may be packaged into kits in a suitable container along with suitable reagents, controls, instructions and the like.

Such peptides or analogs and kits may be used in methods of detection of antibodies to such peptides or analogs present in a sample or active portions or fragments thereof by immunoassay.

Antibodies raised to a peptide can be used in the identification and/or isolation of variant peptides, and then their encoding genes. Thus, the present invention provides a method of identifying or isolating a fibrin degradation product binding epitope (e.g. a fibrin fragment E binding epitope) or variant thereof (as discussed above), comprising screening candidate peptides with a peptide comprising the antigen-binding domain of an antibody (for example whole antibody or a fragment thereof) which is able to bind said fibrin degradation product binding epitope peptide or variant thereof, or preferably has binding specificity for such a peptide. Specific binding members such as antibodies and peptides comprising antigen binding domains of antibodies that bind and are preferably specific for a fibrin degradation product binding epitope peptide or mutant or derivative thereof represent further aspects of the present invention, as do their use and methods which employ them.

Candidate peptides for screening may for instance be the products of an expression library created using nucleic acid derived from an animal of interest, or may be the product of a purification process from a natural source. Analogs may be produced by any of the techniques described herein or may be derived using, for example, combinatorial chemical libraries known in the art. Examples of such libraries are reviewed in Newton GR, Exp. Opin. Ther. Patents (1997) 7(10): 1183-1194.

A peptide found to bind the antibody may be isolated and then may be subject to amino acid sequencing. Any suitable technique may be used to sequence the peptide either wholly or partially (for instance a fragment of the peptide may be sequenced). Amino acid sequence information may be used in obtaining nucleic acid encoding the peptide, for instance by designing one or more oligonucleotides (e.g. a degenerate pool of oligonucleotides) for use as probes or primers in hybridization to candidate nucleic acid, or by searching computer sequence databases, as discussed further herein.

The reactivities of antibodies with a sample may be determined by any appropriate means. Tagging with individual reporter molecules is one possibility. The reporter molecules may directly or indirectly generate detectable, and preferably measurable, signals.

The linkage of reporter molecules may be directly or indirectly, covalently, e.g. via a peptide bond or non-covalently. Linkage via a peptide bond may be as a result of recombinant expression of a gene fusion encoding antibody and reporter molecule.

The mode of determining binding is not a feature of the present invention and those skilled in the art are able to choose a suitable mode according to their preference and general knowledge.

Antibodies according to the present invention may be used in screening for the presence of a peptide or analog, for example in a test sample containing cells or cell lysate as discussed, and may be used in purifying and/or isolating a peptide or analog according to the present invention, for instance following production of the peptide by expression from encoding nucleic acid therefor.

Antibodies of the invention may be used in methods of identifying the active site of the receptor for fibrin fragment E. Antibodies which inhibit the effects of FDPs may be structurally similar to a portion of the active site to which the fibrin fragment E binds and thus may be used to determine the active site of the fibrin fragment E receptor.

Antibodies may modulate the activity of the peptide or analog to which they bind and so, if that peptide or analog has a deleterious effect in an individual, may be useful in a therapeutic context (which may include prophylaxis).

An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

A peptide, analog or antibody according to the present invention may be used in screening for molecules which bind to it or modulate its activity or function. Such molecules may be useful in a therapeutic (possibly including prophylactic) context.

The stimulation of cell proliferation induced by fibrin degradation products, for example fibrin fragment E, provides a target for the development of therapeutic agents capable of inhibiting uncontrolled cell proliferation, for example as is found in restenosis or in tumour cells.

A number of assay formats are described in WO94/10307 and WO96/10425. The provision of the peptides, analogs or antibodies of the invention provide control reagents for such assays which will be desirable in the design of high throughput screening assays for novel compounds which can exert a similar effect. The peptides, analogs or antibodies of the invention further provide a basis for rational drug design of pharmaceutical compounds to target the fibrin fragment E receptor binding site or, for those peptides, the results for which suggest a possible additional or alternative binding site, provide a basis for rational drug design of pharmaceutical compounds to target the alternative or additional binding site.

Peptides, analogs or antibodies of the present invention may be used to develop mimetics. This might be desirable where the peptide, analog or antibody is difficult or expensive to synthesise or where it is unsuitable for a particular method of administration, e.g. peptides may be unsuitable active agents for oral compositions as they may be quickly degraded by proteases in the alimentary canal. Mimetic design, synthesis and testing may be used to avoid randomly screening large numbers of peptides or analogs for a target property.

There are several steps commonly taken in the design of a mimetic from a peptide, analog or antibody having a given target property. Firstly, the particular parts of the peptide, analog or antibody that are critical and/or important in determining the target property are determined. In the case of a peptide, this can be done by systematically varying the amino acid residues in the peptide, e.g. by substituting each residue in turn. These parts or residues constituting the active region of the peptide are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process. A template molecule is then selected onto which chemical groups which mimic the pharmacophore can be grafted. The template molecule and the chemical groups grafted on to it may conveniently be selected so that the mimetic is easy to synthesise, is likely to be pharmacologically acceptable, and does not degrade in vivo, while retaining the biological activity of the lead peptide, analog or antibody. Alternatively, the pharmacophore can be used to form the basis of a search of a computer database of structures to identify a mimetic. The mimetic or mimetics found by any approach described herein can then be screened to see whether they have the target property, or to what extent they exhibit it. Further optimisation and/or modification can then be carried out to arrive at one or more final mimetics for in vivo or clinical testing.

Mimetics obtainable by the above and other methods available in the art form a further aspect of the present invention.

Peptides, analogs or antibodies of the invention may be in a substantially isolated form. It will be understood that the peptide, analog or antibody may be mixed with carriers or diluents which will not interfere with the intended purpose of the peptide, analog or antibody and still be regarded as substantially isolated. A peptide, analog or antibody of the invention may also be in a substantially purified form, in which case it will generally comprise the peptide, analog or antibody in a preparation in which more than 90%, e.g. 95%, 98% or 99% of the peptide, analog or antibody in the preparation is a peptide, analog or antibody of the invention.

Peptides or analogs of the invention may be formulated in the form of a salt. Salts of peptides or analogs of the invention which may be conveniently used in therapy include physiologically acceptable base salts, eg derived from an appropriate base, such as alkali metal (e.g. sodium), alkaline earth metal (e.g. magnesium) salts, ammonium and NR₄ (wherein R is C₁₋₄ alkyl) salts. Salts also include physiologically acceptable acid addition salts, including the hydrochloride and acetate salts.

Peptides (including fusion peptides) analogs, or antibodies of the invention may be formulated into pharmaceutical compositions. The compositions comprise the peptide, analog or antibody together with a pharmaceutically acceptable carrier or diluent. Pharmaceutically acceptable carriers or diluents include those used in formulations suitable for oral, topical, or parenteral (e.g. intramuscular or intravenous) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For example, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the peptide, analog or antibody to blood components or one or more organs.

Suitable liposomes include, for example, those comprising the positively charged lipid (N[1-(2,3-dioleyloxy)propyl]-N,N,N-triethylammonium (DOTMA), those comprising dioleoylphosphatidyl-ethanolamine (DOPE), and those comprising 3β[N-(n′,N′-dimethyl-aminoethane)-carbamoyl]cholesterol (DC-Chol).

Compositions may comprise any desired amount of a peptide, analog or antibody of the invention. In part this will depend upon the intended formulation and its intended use. By way of general guidance the composition may comprise from about 1% to about 99%, for example from 10% to 90% of a peptide, analog or antibody of the invention.

The composition may comprise a mixture of more than one, for example two or three, peptides, analogs or antibodies of the invention.

Peptides, analogs or antibodies of the invention may also be used in conjunction with a second agent capable of modulating cell proliferation.

For example where the peptide, analog or antibody has an inhibitory effect on cell proliferation, it may be used in conjunction with a second agent capable of inhibiting cell proliferation, in order to provide a combined anti-proliferative effect. Thus the composition may also comprise other pharmaceutically active ingredients, in particular cytotoxic and/or cytostatic agents.

Alternatively, a peptide, analog or antibody of the invention may be delivered to a patient in a separate composition from a cytotoxic or cytostatic agent but simultaneously or sequentially. “Sequentially” means that one of the peptides, analogs or antibodies or the agent will be delivered first, and the other delivered within a period of time such that the enhanced effect of the two agents together is achieved in a target proliferating cell. Where one or both agents is delivered over a period of time, e.g. through intravenous infusion, the time period of administration of the agents may be sequential or overlapping.

When used in methods of treatment of the human or animal body, the peptide, analog or antibody and the agent may be administered to a subject at the same site or at different sites.

Thus the invention provides a peptide, analog or antibody of the invention and a cytotoxic or cytostatic agent for separate or simultaneous use in the treatment of proliferating cells, for example tumour cells, either in vitro or in vivo.

Where in vitro use is contemplated, this will include ex-vivo, e.g. in the treatment of bone marrow from a subject which may be reimplanted into the subject after treatment.

The invention further provides the use of a peptide, analog or antibody of the invention for the manufacture of a medicament for the treatment of proliferating cells wherein said cells are also treated, separately or simultaneously, with a cytotoxic or cytostatic agent.

Numerous cytotoxic and/or cytostatic agents are known in the art (e.g. listed in The Merck Index, 12th Edition, 1996) and include:

alkaloids such as etoposide and other toposiomerase inhibitors, paclitaxel, vinblastine and vincristine; alkylating agents such as alkyl sulphonates (e.g. busulfan), aziridines, ethylenimines and methylmelomines (e.g. triethylenemelamine and triethylenephosphoramide), nitrogen mustards (e.g. cyclophosphamide, melphalan and uracil mustard), nitrosoureas and the like;

antibiotics and analogues such as actinomycins, anthramycin, doxorubicin, puromycin and the like;

antimetabolites such as folic acid analogues (e.g. methotrexate), purine analogues (e.g. 6-mercaptopurine and thioguanine) and pyrimidine analogues (e.g fluorouracil);

platinum complexes such as cisplatin; and

other anti-neoplastic compounds including for example hydroxyurea.

In addition, the cytotoxic or cytostatic compound may be an immunomodulatory compound or hormonal analogue compound. Examples of the former include interferons α, β and γ and interleukins such as IL-2. Examples of the latter include antiandrogens, antiestrogens (e.g. tamoxifen), aromatase inhibitors, estrogen analogues, LHRH analogues (e.g. buserelin) and the like.

Cytostatic compounds also include antimetastatic agents such as matrix metalloproteinase inhibitors such as batimastat.

Peptides, analogs, or antibodies of the invention may also be used in methods of treating a number of diseases, in particular those diseases in which uncontrolled proliferation of cells may play a part. Conditions in which uncontrolled cell proliferation may be treated include vascular restenosis, cancer, atherogenesis, rheumatoid arthritis, complications of diabetes such as retinopathies, renal diseases and psoriasis.

Angiogenesis and Wound Healing

Fibrin deposition and lysis are essential features of normal wound healing processes, as demonstrated by abnormalities of healing in the plasminogen and fibrinogen knockout mouse models (5, 6). The present inventors have shown in a mouse incised wound model that the peak of angiogenic activity in simple wound extracts occurs at day 3, preceding the peak of wound vascular density at day 5 (7) with the bulk of the angiogenic activity suggested to be attributable to fibrin fragment E (8). Compounds of the invention may therefore find use in treatments concerned with wound healing.

As described in the examples peptides, some peptides of the invention, for example WTM35 and WTM36, appear to have stimulatory effects on cell proliferation which does not compete with that of FDPs i.e. work via a different mechanism. Stimulatory peptides and their analogs may therefore be useful in treatments related to wound healing and ulcers, where sustained growth is required without interference with the effects of fibrin or FDPs per se. Equally they may be useful e.g. when used topically, in conjunction with systemic medicaments which inhibit any of these processes, such as cancer therapies, in order to counteract the effects of those medicaments in areas or tissues in which it is desired to do so.

In some aspects of wound healing, it is desirable to have some control over the rate and degree of scar tissue formation and deposition. This is particularly important in patients undergoing plastic surgery. Certain peptides of the invention, in particular WTM34 has been shown to have an inherent inhibitory effect on cell proliferation. Such peptides and their analogs may therefore find use in in treatments for controlling or reducing scarring, for example scarring associated with plastic surgery.

Vascular Restenosis

The likelihood of clinically significant post-angioplasty restenosis has been generally understood to be predicted to a fair extent by the amount of blood clot at the angioplasty site. The antithrombin drug hirudin has been partially successful in reducing restenosis experimentally and in man (9), but it has been unclear whether this is attributable to clot reduction or prevention of direct thrombin stimulation of smooth muscle cell proliferation via the PAR-1 thrombin receptor (10). However it is now apparent that the PAR-1 knockout mouse is normal and has near normal wound healing (11), and that intimal hyperplasia is not inhibited by an antisense thrombin receptor oligodeoxynucleotide following carotid injury in the rabbit (12). An alternative candidate that is both thrombin and plasmin dependent, but not thrombin receptor dependent, is fibrin fragment E. Fibrin degradation products are abundant at sites of healing and repair, including sites of vascular injury and in extracts of proliferative types of human atherosclerotic plaque. The present inventors have shown that fibrin fragment E stimulates smooth muscle cell proliferation and outgrowth from aortic media explants in culture (13). This occurs in serum rich culture in which thrombin is inactive. The peptides, analogs and antibodies therefore find use in treatment and prevention of restenosis.

Atherogenesis

The link between abnormalities of the coagulation system and thrombosis of a coronary artery is well established not only in terms of the actual clot but in terms of risk of myocardial infarction in human populations. In many prospective studies, the plasma levels or activities of coagulation factors such as fibrinogen, factor VIIa and fibrin degradation products (D dimer assays) have been shown to be predictive risk factors for myocardial infarction, and other vascular events such as stroke and progressive peripheral vascular disease (14, 15, 16). A substantial proportion of the risk due to cigarette smoking is attributable to raised fibrinogen. The mode of interaction of this risk factor with the actual lesions of atherosclerosis, the atherosclerotic plaques within the artery wall, remains unexplained, but the end result, accumulation of fibrin admixed with the lipid core of the plaque and forming overlying thrombus in minor and major terminal events is well established.

The major pathogenic feature of atherogenesis is the response to fibrin and lipid accumulation by the smooth muscle cells of the arterial wall. Smooth muscle cell proliferation has long been recognised as the key event in plaque development, as it is in the more acute lesion of post angioplasty restenosis (17). Atherosclerotic lesions can be divided into several types, the earliest thought to be the gelatinous lesions, the precursors of the fibrous plaques. The early gelatinous lesions contain little lipid but significant amounts of fibrin related antigens (FRA) (18). In the more advanced lesions, fibrin is deposited in layers suggesting repeated thrombotic episodes (19). In all lesions the FRA are largely derived from cross-linked fibrin not fibrinogen suggesting continuous deposition and lysis of fibrin (20).

Soluble extracts of intima from active types of lesions from human autopsy and surgical material have been shown by the present inventors to stimulate cell proliferation in the in vivo chick chorioallantoic membrane test model (21). This work was extended to show that for a short series of stimulatory extracts, the bulk of the activity was removed by passing each through an affinity column containing antifibrinogen antibody (22). Selective removal was again achieved with a bound specific anti fragment E antibody, but not with a bound anti fragment D antibody.

Rheumatoid Arthritis

Rheumatoid arthritis is of unknown cause but is known to be driven by the immune system. This causes episodic inflammation of the synovial lining of the joint, with deposition of fibrin which becomes organised by fibrovascular ingrowth, termed pannus, forming a membrane rich in inflammatory cells. This extends over the joint cartilage, releasing proteolytic enzymes that digest and gradually destroy the joint surface causing pain and immobility. Anti inflammatory drugs help symptoms but do not arrest disease progress significantly. Prevention of pannus extension during acute episodes may well be advantageous.

Diabetic retinopathy

Diabetic retinopathy is due to narrowing of the microvascular blood supply vessels within the eye as elsewhere in the body in poorly controlled diabetes mellitus. It is characterised by proliferation of leaky new blood vessels in response to ischaemic areas of retina. The combination of fibrovascular proliferation and contraction involves the vitreous and distorts and destroys the retina. Fibrin deposition and degradation are likely to contribute to this disadvantageous instance of normal healing and repair.

Renal Disease

Acute and focal glomerulonephritis are characterised by deposition of both immune complexes and fibrin in the glomerulus with associated inflammatory cells. There is resultant cellular proliferation leading to glomerulosclerosis and permanent loss of function with renal failure in many cases. Anti-inflammatory drugs could be supplemented with the agents to inhibit cell proliferation.

Tumour Growth and Metastasis

For a malignant, invasive epithelial tumour of any type to grow more than 1 mm and invade surrounding normal tissues, there is an absolute requirement for recruitment of a new blood supply. This phenomenon of tumour angiogenesis provides a target for therapeutic anti cancer intervention. These new small capillary vessels are leaky, and plasma proteins including fibrinogen are abundant in adjacent connective tissue at the tumour edge. Although it was once believed that fibrin was deposited at the moving edge of stroma surrounding most invasive tumours as in a wound (23), it is now believed that many tumour types do not display a complete set of procoagulant factors for this to happen. However there are two major exceptions, oat cell carcinoma of lung and clear cell carcinoma of kidney (24, 25). These two tumour types are common, are extremely vascular, show fibrin deposition, and are hard to treat once spread by metastases from the site of origin has occurred. Some modest improvement in survival has been shown in a trial of terminally ill patients with oat cell carcinoma with the drug warfarin which inhibits clotting (26). One problem with such drugs is that complete inhibition is never achieved because of very real risks of major haemorrhage. Inhibition of the fibrin E stimulatory contribution not to tumour growth but to tumour angiogenesis may be a useful adjunct to the current partially effective treatments by chemotherapy and radiotherapy. Other tumour cells which may be used as a target include cells of solid tumours such as lung (including small cell lung), bowel (colon), breast, ovarian, prostate, stomach, liver, pancreatic and skin tumours, as well as leukaemias.

Formulation and administration

In all these examples of disease, the clinical aim would be to prevent the initiating cause but this is often not possible. Selective inhibition of angiogenesis and cell proliferation would be highly desirable. At the same time, it would be preferable to avoid interference with other aspects of the normal inflammatory response, and with blood clotting and fibrinolysis. However clinical manipulation at one site may conflict with clinical problems at other sites. For example, systemic administration of an antiangiogenic drug to inhibit tumour growth may adversely affect normal healing and repair of a healing skin wound, or a peptic ulcer of stomach.

The severity of potential limitations depends on the therapeutic margin of treatment success over unwanted side effects. Short term treatment and localised treatment offer solutions to some problems but not all. It is therefore envisaged to utilise protective or therapeutic treatments at the other sites and conditions at risk of collateral damage. Therefore, treatments may be developed whereby cell proliferation and/or angiogenesis inhibitors may be used at the site of interest or systemically but locally protective FDP induced cell proliferation and/or angiogenesis agonists are provided at other sites that stimulate angiogenesis and the normal cell proliferative response. Combinations of systemic and local administration may become feasible and effective.

In general, the methods of the invention will involve administering to a patient in need of treatment an effective amount of a peptide, analog or antibody (or composition thereof) of the invention. Suitable routes of administration of compounds of the invention include oral or parenteral, and will depend in part upon the intended use and the discretion of the physician. Small peptides may be administered orally although parenteral administration may generally be more convenient in some circumstances.

The amount of peptides, analogs or antibodies of the invention administered to a patient is ultimately at the discretion of the physician, taking account of the condition of the patient and the condition to be treated.

Doses may be administered continuously, e.g in the form of a drip, or at discrete intervals, e.g twice daily, daily, weekly or monthly. Doses may also be administered topically to achieve concentrations of active agent on the skin in the ranges described above.

Where the peptide, analog, antibody of the invention is being used for the treatment of ulcers of, for example the skin or the eye, or in the prevention or treatment of scarring, for example associated with plastic surgery, it will preferably be provided in a composition suitable for topical application.

Preparations for topical administration may be in the form of ointments, creams, gels, lotions, pessaries, aerosols or drops (e.g. eye drops). Ointments and creams may, for example, be formulated with an aqueous or oily base with the addition of suitable thickening and/or gelling agents.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

Where a peptide, analog or antibody of the invention is to be administered in conjunction with a cytotoxic or cytostatic agent, the dose of said agent will be in accordance with manufacturers' instructions.

Peptide, analogs or antibodies may be selectively directed to tumour cells by various mechanisms in order to enhance their effectiveness and to avoid effects on normal cells. Such mechanisms include coupling the peptide, analog or antibody to molecules which specifically interact with receptors or antigens on target cells, such as VEGF receptors or CEA. Alternatively gene therapy vectors expressing peptides of the invention may comprise an expression system whose promoter is selectively activated in tumour cells, such as promoters active in fetal liver cells.

In a further embodiment, a peptide, analog or antibody of the invention may be incorporated into a stent which is introduced into the arteries of a patient during an angioplasty procedure. This is in order for the peptide, analog or antibody of the invention to treat restenosis. The stent is a hollow metal tube, usually made of stainless steel and optionally coated with a polymeric material such as a plastic which is expanded during the procedure so as to be left in place in the artery to treat heart disease caused by arterial narrowing. A problem with this procedure is the occurrence of restenosis, i.e. the smooth muscle cells tend to grow back and further treatment is ultimately required. By coating the stent with a peptide, analog or antibody of the invention, the peptide, analog or antibody is delivered locally into the vessel wall and will prevent local regrowth of cells by inhibiting stimulation of cell proliferation by FDPs.

Peptides, analogs or antibodies of the invention may be either coated onto or incorporated into the stent by conventional means known per se in the art. For example, the peptides, analogs or antibodies may be mixed with a pharmaceutically acceptable carrier compatible with the stent material and coated on or into the stent. Where incorporation into the stent is contemplated it is desirable that the stent comprises an open celled polymeric structure. Where the stent is in the form of a mesh, the peptides, analogs or antibodies may be incorporated into a suitable delayed release carrier contained in the spaces between the mesh strands. Delayed release formulations are widely available for a number of different purposes in the art; these include formulations based on pharmaceutically acceptable polymers which dissolve slowly in the body following implantation.

A number of coronary stents have been approved for clinical use in the USA by the FDA. These include balloon expandable stents such as the Palmaz-Schatz stent made by Cordis Corporation (a division of Johnson & Johnson Interventional Systems) and the Gianturco-Roubin II (GR-II) stent made by Cook Cardiology (Bloomington, Ind., USA). Self-expanding stents are also used in the art, e.g. the Wallstent (Medinvent-Schneider, Switzerland). Generally these stents are made of a wire of around 0.1 mm (e.g. from 0.07 to 1.5 mm) diameter, are designed to expand to a diameter of 3-5 mm, and are around 10 to 20 mm in length.

Examples of stent coatings to which reference may be made for the provision of peptide coated stents of the invention include a heparin-coated Palmaz-Schatz stent (Serruys et al, Circulation, 1996, 93;412-422) and a platelet glycoprotein IIa/IIIa receptor antibody polymer-coated stent (Aggarwal et al, Circulation, 1996, 94;3311-3317).

For further guidance, those of skill in the art may also make reference to “Coronary Artery Stents”, an ACC Expert Consensus Document (Pepine et al, J. Am. Coll. Cardiol., 1996, 28;782-794.

Another possible use of a peptide, analog or antibody of the invention is to modify fibrin glues. Modification of fibrin glues to promote or inhibit the cell proliferation induced by fibrin degradation products can be achieved by preparation and admixture with modulators (promoters or inhibitors) of the site of interaction of fibrin E as provided by the present invention. These glues are increasingly used for a wide variety of operations where sutures are impractical.

The following examples illustrate the invention.

### DESCRIPTION OF THE DRAWINGS

FIG. 1 illustrates the effect of antibodies to peptides WTM33, WTM34, WTM35, WTM36 and WTM37 on the stimulatory effect of FDP. Student's t test shows a significant (P<0.05) inhibition of the increase in DNA synthesis compared with the FDP only control group. The stimulation measured with antibodies to WTM33, WTM34, WTM35, WTM36 and WTM37 are significantly different from the FDP group, but not from the control group.

FIG. 2 illustrates the effect of a mixture of antibodies to peptides WTM250, WTM257 and WTM260 on the stimulatory effect of FDP. Student's t test shows that the presence of antibodies to these peptides had no significant effect on the FDP induced stimulation. The stimulation was, however, significantly different from that measured in the absence of FDP in the negative control.

FIG. 3A illustrates a direct comparison of the effect of peptide WTM 35 (2.04 μM-16.32 μM, ______) on cell proliferation relative to cell proliferation in control culture medium measured using the chick CAM assay with the effect of fibrin degradation products (- - - -).

FIG. 3B illustrates the effect of peptide WTM 35 (2.04 μM-16.32 μM) on cell proliferation relative to control in the presence (______) and absence (- - - -) of 5 μM FDPs.

FIG. 3C illustrates the stimulatory effect of peptide WTM 35 (2.04 μM -16.32 μM) in the presence of 5 μM FDPs on cell proliferation relative to control compared to the stimulatory effect of 5 μM FDPs alone. Column 1: 2.04 μM WTM35, Column 2: 4.81 μM WTM35, Column 3: 8.16 μM WTM35, Column 4: 10.88 μM WTM35, Column 5: 16.32 μM WTM35, and Column 6: 5 μM FDPs alone.

FIG. 4A illustrates a direct comparison of the effect of peptide WTM 36 (2.04 μM-16.32 μM, ______) on cell proliferation relative to cell proliferation in control culture medium measured using the chick CAM assay with the effect of fibrin degradation products (- - - -).

FIG. 4B illustrates the effect of peptide WTM 36 (2.04 μM-16.32 μM) on cell proliferation relative to control in the presence (______) and absence (- - - -) of 5 μM FDPs.

FIG. 4C illustrates the stimulatory effect of peptide WTM 36 (2.04 μM-16.32 μM) in the presence of 5 μM FDPs on cell proliferation relative to control compared to the stimulatory effect of 5 μM FDPs alone. Column 1: 2.04 μM WTM36, Column 2: 4.81 μM WTM36, Column 3: 8.16 μM WTM36, Column 4: 10.88 μM WTM36, Column 5: 16.32 μM WTM36, and Column 6: 5 μM FDPs alone.

FIG. 5A illustrates a direct comparison of the effect of peptide WTM 37 (2.04 μM-16.32 μM, ______) on cell proliferation relative to cell proliferation in control culture medium measured using the chick CAM assay with the effect of fibrin degradation products (- - - -)

FIG. 5B illustrates the stimulatory effect of peptide WTM 37 (2.04 μM-16.32 μM) in the presence of 5 μM FDPs on cell proliferation relative to control compared to the stimulatory effect of 5 μM FDPs alone. Column 1: 2.04 μM WTM37, Column 2: 4.81 μM WTM37, Column 3: 8.16 μM WTM37, Column 4:10.88 μM WTM37, Column 5: 16.32 μM WTM37, and Column 6: 5 μM FDPs alone.

FIG. 6 illustrates a direct comparison of the effect of peptide WTM 33 (2.04 μM-16.32 μM, ______) on cell proliferation relative to cell proliferation in control culture medium measured using the chick CAM assay with the effect of fibrin degradation products (- - - -).

FIG. 7A illustrates a direct comparison of the effect of peptide WTM 34 (2.04 μM-16.32 μM) on cell proliferation relative to cell proliferation in control culture medium measured using the chick CAM assay with the effect of fibrin degradation products (- - - -).

FIG. 7B illustrates the effect of peptide WTM 34 (2.04 μM-16.32 μM) on cell proliferation relative to control in the presence (______) and absence (- - - -) of 5 μM FDPs.

FIG. 8A illustrates the effect of peptide WTM33 (0-60 μg/ml) on the number of vessels per mm of CAM.

FIG. 8B illustrates the effect of peptide WTM34 (0-60 μg/ml) on the number of vessels per mm of CAM.

FIG. 8C illustrates the effect of peptide WTM35 (0-60 μg/ml) on the number of vessels per mm of CAM.

FIG. 9 compares performance of WTM33 in primary tumour model with a cyclophosphamide.

FIG. 10 compares the performance of WTM34 in metastatic tumour model with +ve and −ve controls.

FIG. 11 shows effect of five fibrin E peptide analogues on weight of lungs of mice injected with LLC in the metastatic tumour model (increased lung weight being due to metastatic tumour masses). Performance of the 5 fibrin E peptides in metastatic tumour model is expressed as increase in lung mass over control. Mice that were not injected with LLC cells did not develop lung metastases.

### EXAMPLES

In examples 1 and 2, it is shown that fibrin fragment E binds to a cell membrane component, indicating that a cell membrane receptor with specificity for fibrin fragment E is present and that antibodies to fibrin fragment E block FDP induced stimulation of cell proliferation. Examples 3 and 4 relate to antibodies to peptides of the invention which are shown to have modulatory effect on FDP-induced stimulation of cell proliferation. Example 5 relates to peptides of the invention which are shown to have modulatory effects on the stimulation of cell proliferation. Examples 6 and 7 relate to peptides of the invention which are shown to have modulatory effects on the formation of vessel numbers in CAM, or on tubules resembling microvessels by human endothelial cells in an in vitro model of angiogenesis. Example 8 relates to peptides of the invention which are shown to have modulatory effects on the formation of tumours in a mouse model.

### Example 1

Fibrin Fragment E Binds to a 66 kDa Cell Membrane Component

i) Digoxygenin Labelling of Fibrin Fragment E

327 mg of digoxygenin ester, dissolved in 8.18 ml DMSO was added to 1 mg of fibrin fragment E in 1 ml of phosphate buffered saline (PBS) and incubated at room temperature for 2 hours. This was then dialysed against PBS (3 changes of 11) to remove the unreacted ester and DMSO. Detection of the labelled protein was then carried out using anti-digoxygenin alkaline phosphatase raised in sheep (Boehringer).

ii) Ligand blot

Chick fibroblasts, Cos7, mouse 3T3 and human embryonic lung cells (HEL) were cultured in a 25 cm² flask (Nunc) until confluent (approximately 3×10⁶ cells). The cells were then floated off the flask using PBS with 2% EDTA and slight scrapping with a rubber policeman. The cells were centrifuged for 5 min at 3,000 rpm and resuspended in hypotonic shock solution, sonicated for 30 min and then centrifuged and resuspended in 1 ml of PBS. 100 ml of the cells was then added to 35 ml of SDS containing gel loading dye (8% SDS, 40% glycerol, 12.5% 0.5M Tris-glycine buffer pH 6.8 and 10 mg of bromophenol blue), and heated to 95° C. for 5 min.

50 ml of the cells in loading dye were applied to gradient polyacrylamide gels (3-20%) and electrophoresed for 5 hours until the dye reached the base of the gel. The gel was then washed several times in Tris buffered saline containing 0.5% Triton-X-100 to remove the SDS. The gel was then blotted on to PVDF membrane (Millipore) using a Bio-Rad blotting system in Tris glycine buffer (25 mM Tris and 192 mM glycine).

The blotting membrane was blocked using 5% bovine serum albumin in Tris buffered saline (TBS). The blot was then incubated overnight at 37° C. with digoxygenin labelled fibrin fragment E (100 mg in 100 ml of TBS). The membranes were washed three times in TBS 0.5% Tween 20 for 10 minutes and incubated for 1 hour with sheep anti-digoxygenin antibody conjugated to alkaline phosphatase. The membrane was washed, 3 times, again with TBS Tween followed by development with nitro blue tetrazolium, bromo-chloro-indolyl-phosphate in sodium bicarbonate buffer (NaHCO₃ 100 mM, MgCl₂ 10 mM) to visualise the position of the receptor containing band. For each cell type, a band was identified of approximately 66 kDa. To confirm this finding, membrane immunoblot experiments were performed using cells previously challenged with fibrin fragment E as described below.

iii) Membrane Immunoblotting

Chick fibroblasts, Cos7, mouse 3T3 and human embryonic lung cells (HEL) were cultured in a 25 cm² flask (Nunc) until confluent (approximately 3×10⁶ cells). 100 mg of digoxygenin labelled fibrin fragment E was added to the cells and incubated at 37° C. Cells were harvested by rinsing with PBS and then floated off the flask using PBS with 2% EDTA and slight scraping with a rubber policeman. The cells were centrifuged for 5 min at 3,000 rpm and resuspended in hypotonic shock solution for 15 min, centrifuged and resuspended in 1 ml of PBS. 100 ml of the cells was then added to 35 ml of SDS containing gel loading dye (8% SDS, 40% glycerol, 12.5% 0.5M Tris glycine buffer, pH 6.8 and 10 mg of bromophenol blue), and heated to 95° C. for 5 min.

50 ml of the cells in loading dye were applied to gradient polyacrylamide gels (3-20%) and electrophoresed for 5 hours until the dye reached the base of the gel. The gel was then washed several times in Tris buffered saline containing 0.5% Triton X to remove the SDS. The gel was then blotted on to PVDF membrane (Millipore) using a Bio-Rad blotting system in Tris glycine buffer (25 mM Tris and 192 mM glycine).

The blotting membrane was blocked using 5% bovine serum albumin in Tris buffered saline (TBS). The blot was then incubated overnight at 37° C. for 1 hour with rabbit anti fibrinogen antibody (Dako). The membranes were washed three times in TBS 0.5% Tween 20 for 10 minutes and incubated for 1 hour with goat anti rabbit antibody conjugated to alkaline phosphatase. The membrane was washed, 3 times, again with TBS Tween followed by development with nitro blue tetrazolium (NBT), bromo-chloro-indolyl-phosphate (BCIP) in sodium bicarbonate buffer (NaHCO₃ 100 mM, MgCl₂ 10 mM pH 9.8). This resulted in a band of approximately 122 kD, which is consistent with fragment E (55 kD) bound to a membrane fragment of 66 kD. Further evidence of binding to a receptor was provided by cell immunochemistry and binding assays as described below.

iv) Cell Immunohistochemistry

Chick fibroblasts were cultured in a 25 cm² flask (Nunc) until confluent (approximately 3×10⁶ cells). The cells were washed in PBS and then trypsinised and diluted to 3×10⁵ cells per ml in Dulbecco modified medium with 10% foetal calf serum. 50 ml of the cells suspension was plated into each well of a Nunc culture well slide. The cells were incubated overnight to allow adhesion and recovery from the passaging. Digoxygenin labelled fibrin fragment E was added to a final concentration of 8 mg per ml. Control wells contained unconjugated digoxygenin and PBS.

The cells were rinsed after 2 hours with PBS then washed 3 times in TBS followed by one wash in distilled water. The cells were then incubated for 2 hours with sheep anti-digoxygenin alkaline phosphatase. The slides were then washed three times in TBS and developed with NBT and BCIP in bicarbonate buffer pH 9.8. The slides were air dried and then mounted for observation of membrane staining under the microscope. The cells were shown to bind fibrin fragment E.

v) Cell Binding Assays

Chick fibroblasts, Cos7, mouse 3T3 and human embryonic lung cells (HEL) were cultured in a 25 cm² flask (Nunc) until confluent (approximately 3×10⁶ cells). The cells were trypsinised and resuspended at concentration of 1.3×10⁵ cells/ml. 200 μl of this cell suspension was added to each well of a 96 well culture plate (Nunc). The cells were incubated overnight at 37° C. to allow adherence and recovery. The cells were then divided into three sections, digoxygenin controls, PBS controls and digoxygenin labelled fragment E tests. Increasing concentrations of digoxygenin and digoxygenin labelled fibrin fragment E were applied from 0-30 mg per well. The plates were incubated for 6 hours at 37° C. under culture conditions.

The plates were emptied of media and washed three times with PBS. The cells were then incubated for 2 hours with sheep antidigoxygenin alkaline phosphatase and developed with p-nitrophenyl phosphate in bicarbonate buffer pH 9.8. The plates were read on a Titertek Multiscan plate reader at 405 nm. For each of the cell types, high absorbance was demonstrated compared to the control preparations, indicating binding of the digoxygenin labelled fibrin fragment E to the cells.

The demonstration that fibrin fragment E binds to each of the cell types tested suggests that a fibrin fragment E specific receptor of 66 kDa is present on the cell membrane.

### Example 2

Antibodies to Fibrin Fragment E block FDP Induced Stimulation of Cell Proliferation.

i) Polyclonal Antibody production

Polyclonal antibodies were raised to fibrin fragment E using the following immunisation protocol.

Rabbits and rats were immunised with fibrin fragment E, made by thrombin treatment of fibrinogen fragment E (Diagnostica Stago). 50 μg of fibrin fragment E in 0.5 ml of PBS was mixed with 0.5 ml of Freunds complete adjuvant. The rabbits were immunised intramuscular and the rats intraperitoneal. The animals were then boosted at 6 and 12 weeks later with 50 μg of a long fibrin digest, which was shown to contain only fibrin fragment E, in incomplete Freunds adjuvant. The animals were then bled and the antisera tested for reactivity on blots of fibrin degradation products.

ii) CAM Cell Proliferation Assay

Cell proliferation was measured using the chick chorioallantoic membrane (CAM) model as described previously (4, 29, 30).

Briefly, on day zero, fertile hens' eggs, are placed in an egg incubator 37.5° C. with pointed ends facing downwards. On day 3, using a 2 ml syringe with a 21G needle, 2 ml of albumen is removed and the eggs returned to the incubator. On day 4, each egg is punctured by a needle at the blunt end into the air space. A square of shell is then removed, causing the egg contents to drop into the space left by collapse of the air space at the end of the egg and the withdrawal of albumen. The volume of the space above the dropped CAM is approximately 7 ml. The square opening in the shell is covered with Sellotape (RTM). The eggs then have to be kept level in the incubator without tilting. On day 10, the surviving eggs are organised into groups of preferably no less than 10. Test and control samples are prepared and each egg receives 0.3 ml of sample injected through the Sellotape (RTM) onto the upper surface of the CAM.

The technique of removal of each CAM is simple but crucial to the rationale of achieving results expressed as total DPM per CAM. By 20 min, methyl-[³H]thymidine is incorporated only into the CAM which has been directly exposed. Beyond 30 min, [³H]thymidine becomes detectable in adjacent CAM that is applied to the shell, via uptake from the bloodstream. Each CAM is removed by holding the egg in the palm of one hand, Sellotape window face down, and piercing the side with pointed scissors well above the level of the dropped CAM. Scissors are used to cut round the shell, and the residual yolk and embryo tipped out into a disposal container once the umbilical vessels connecting the embryo to the CAM are cut. Each CAM is rinsed briefly in saline, blotted dry and placed in a 20 ml Sterilin (RTM) plastic disposable container in 5 ml of distilled water. On day 11 after 18 h, which is optimal for wound extracts and fibrin degradation products, the assay for measurement of DNA synthesis is performed. Methyl-[³H]thymidine is applied onto the CAM surface using 0.5 ml of 2 μCi/ml normal saline per egg. (Methyl-[³H]thymidine is used to avoid recycling of thymidine). After 20 min the eggs are removed to a radioactive type bench and all eggs are rapidly injected through the Sellotape window with 5 ml of ice-cold normal saline to halt metabolism.

On Day 12, each CAM is homogenised briefly in the 5 ml of H₂O using for example a homogeniser with a suitable head. 5 ml of 10% trichloroacetic acid (TCA) is added to each, vortexed and centrifuged at approximately 1,000 g for about 8 min×3. The precipitate is resuspended finally in 2.5 ml of H₂O. Scintillation vials are filled with 5 ml of thixotropic scintillation fluid and vortexed and the contents immediately poured into a vial. Each vial is vortexed until the contents become a gel. To reduce chemiluminescence, the vials are stored at 4° C. overnight in the dark. The tissue debris becomes efficiently dispersed throughout the thixotropic scintillant as a fine emulsion suitable for counting. Nevertheless there is considerable loss of efficiency due to colour, salt and protein quenching inherent to the nature of the tissue, and necessitates accurate quench correction curves created by, for example, the LKB “Hat-trick” method that can be used to check a real CAM sample. This method copes with the relatively large amount of proteinaceous material derived from each CAM.

By applying this assay to the whole “dropped” area of CAM exposed to test or control substances, the results can be expressed per CAM, independent of changes that may be induced in weight and protein content due to, for example, oedema, and of changes in cellularity due to inflammatory cell influx.

The assay used is based on quantitative measurement of DNA synthesis in the CAM after exposure to control and test substances 18 h after application in liquid form to the whole “dropped” area of each CAM (4,29). This assay is a measure of changes in CAM vascularity (30).

The rabbit and rat antisera were mixed with active fibrin degradation products. These were applied to the chick CAM in the presence of FDPs. A positive control (fibrin degradation products) and a negative control (the antibody alone) were also tested. The CAM was processed for incorporation of tritiated thymidine. Results showed that the fibrin degradation products were active and that the rat and rabbit antisera to fibrin fragment E removed the activity.

### Example 3

Synthesis of Peptides

The peptide sequence of the fibrinogen β chain is known and has Genbank accession no. M64983. The present inventors have designed and synthesised the following peptides corresponding to the fibrin/ogen β chain 28-42, 66-80, 81-95, 96-110, 111-121, 15-27, 54-64 and 43-53 sequences:

| | | |
|---|---|---|
| SLRPAPPPISGGGYR | (SEQ ID NO: 1) | (WTM33) |
| LHADPDLGVLCPTGC | (SEQ ID NO: 2) | (WTM34) |
| QLQEALLQQERPIRN | (SEQ ID NO: 3) | (WTM35) |
| SVDELNNNVEAVSQT | (SEQ ID NO: 4) | (WTM36) |
| SSSSFQYMYLL | (SEQ ID NO: 5) | (WTM37) |
| GHRPLDKKREEAPC | (SEQ ID NO: 6) | (WTM250) |
| KVERKAPDAGGC | (SEQ ID NO: 7) | (WTM257) |
| ARPAKAAATQKC | (SEQ ID NO: 8) | (WTM260) |

Note that the numbering for the β chain relates to numbering for the fibrinogen chain and that the cysteine residue at the end of each of peptides WTM250, WTM 257 and WTM260 (SEQ ID NOS: 6, 7 and 8 respectively) does not appear in the wild-type fibrin/ogen sequence. Peptides WTM250, WTM 257 and WTM260 correspond to regions of the fibrin/ogen molecule at which plasmin cleaves the molecule. It was hypothesised that these regions would be exposed and thus may represent potential active sites of the molecule.

### Example 4

i) Polyclonal Antibody Production

Polyclonal antibodies were raised to peptides WTM 33, WTM 34, WTM 35, WTM 36, WTM 37, WTM 250, WTM 257 and WTM 260 using the following immunisation protocol for each peptide individually. Rabbits were immunised with the peptide. 50 g of the peptide in 0.5 ml of PBS was mixed with 0.5 ml of Freunds complete adjuvant. The rabbits were immunised intramuscularly. The animals were then boosted at 6 and 12 weeks later with 50 μg of the peptide in incomplete Freunds adjuvant. The animals were then bled and the antisera tested for reactivity on blots of fibrin degradation products.

ii) Antibodies Raised to WTM33, WTM34, WTM35, WTM36 and WTM37 Inhibit Cell Proliferation.

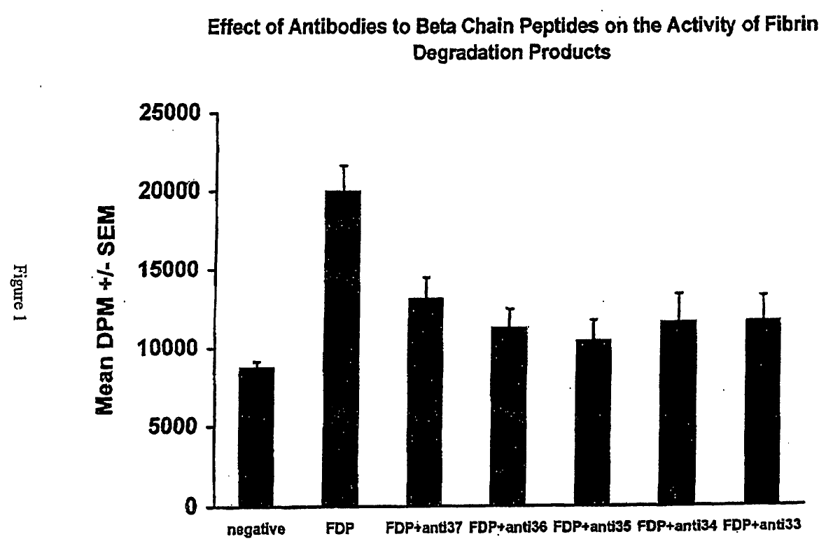

Antibodies raised to each of WTM33, WTM34, WTM35, WTM36, WTM37, WTM250, WTM257 and WTM260 were tested in the chick CAM assay as described above. As shown in FIG. 1, in the presence of any one of anti-WTM33, anti-WTM34, anti-WTM35, anti-WTM36 or anti-WTM37 antiserum at 150 μl/ml, stimulation of cell proliferation by 200 μg/ml FDPs was inhibited.

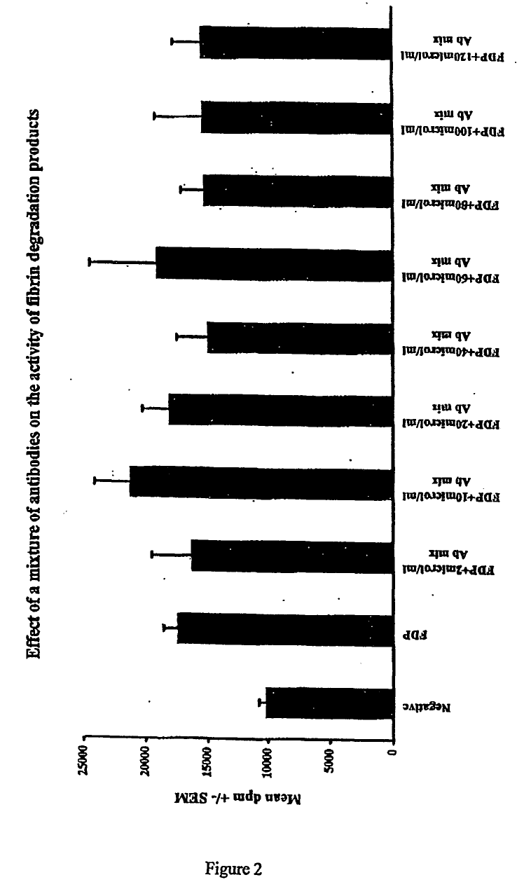

In contrast no such effect was seen using any of the antiserum raised to WTM250, WTM257 or WTM260, each at 20 μl/ml (data not shown). As explained above, from the position of these peptide sequences in the intact molecule, it was expected that these peptides would be likely to represent active sites and therefore the antiserum to such peptides would inhibit stimulation. The lack of inhibition of the FDP induced stimulation observed with the antisera to WTM250, WTM257 or WTM260 was therefore unexpected. In order to address the possibility that this lack of inhibition was simply a result of the concentration being too low, the assay was repeated using an admixture of WTM250, WTM257 and WTM260 antiserum over a range of combined concentrations from 2 μl/ml to 120 μl/ml. As shown in FIG. 2, no inhibition of FDP-induced cell proliferation was observed at any concentration tested.

The sequence information for the finally selected epitopes, derived from analysis of the sequences of the peptides of the invention can be used not only to locate the active site on the molecule, and to perpetuate blocking antibodies, but also to synthesise large quantities of short peptides and short peptide analogues. These can be tested for competitive blocking activity for fibrin fragment E. Such peptides and analogs are potential therapeutic agents in the longer term for blocking the cell stimulatory effects of fibrin fragment E in vivo in a potentially wide variety of pathologies (30) without the attendant risks of interfering in clotting or fibrinolysis. Previous work of the present inventors has shown that admixture of blocking antisera to fibrin E will inhibit the angiogenic effect of experimental mouse wound extracts (31) and extracts of proliferative types of human atherosclerotic plaques (21). Sustained delivery in vivo to inhibit, for example, wound healing should be more readily achieved by administration of small peptides than polyclonal antisera from another species.

### Example 5

i) Peptide WTM 35 Stimulates Cell Proliferation

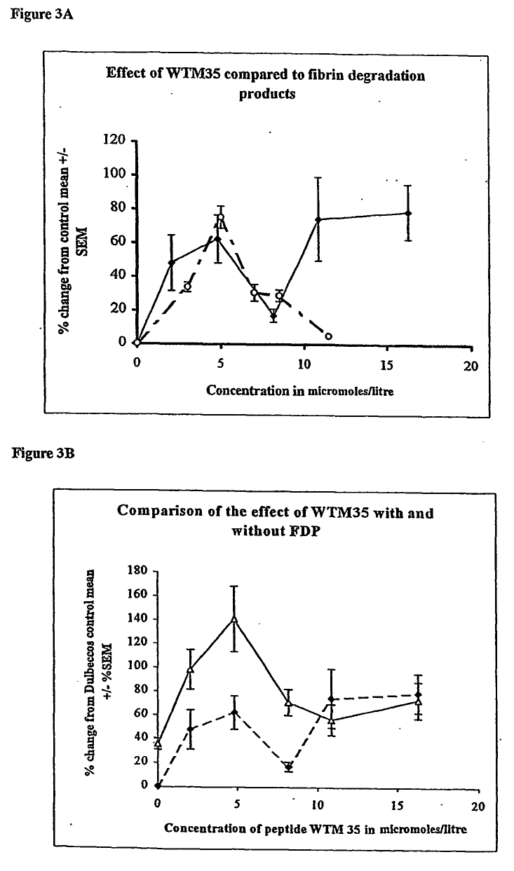

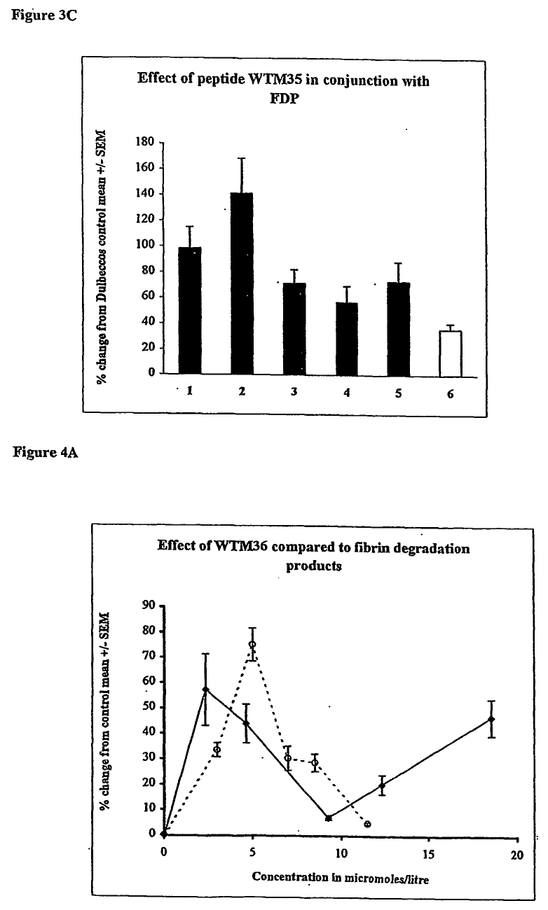

The WTM35 peptide was tested for modulation of cell stimulation using the chick CAM assay as described above. The results are shown in FIGS. 3A, 3B and 3C. In FIG. 3A, the stimulatory effects of WTM35 across a range of concentrations (2.04 μM-16.324 μm) was compared with the stimulatory effects of FDPs at the same concentrations. In each case, the stimulatory effect is shown relative to the results obtained using a Dulbecco's PBS negative control. As shown in the figure, the maximum stimulatory effect achieved with FDPs occurs at 5 μM with higher concentrations of FDPs having progressively reduced stimulatory effects. This is consistent with previous studies on the effects of FDPs, which show no recovery of stimulatory activity at higher concentrations. Without being limited by one particular theory, previous work of the inventors has suggested that the reduction in the stimulatory effect seen with increasing concentrations is due to internalisation of the receptor complex at high concentrations of FDPs.

WTM35 has a similar effect on cell proliferation over a concentration range 2.04 μM-8.162 μM. However, in contrast to the results obtained with FDPs, at higher concentrations, WTM35 has a further cell proliferative effect. On repeating the assay with the same concentrations of WTM35 in the presence of 5 μM FDPs, the stimulatory effect was greater still with no apparent competition with the effect exhibited by the FDPs. Indeed, the effect appears to be additive (FIG. 3B). FIG. 3C compares the stimulatory effect of WTM35 (2.04-16.324 μM) in the presence of 5 μM FDPs with that of 5 μM FDPs alone. At all concentrations, WTM35 does not appear to compete with the stimulatory effect of the FDPs.

The results obtained with WTM35 suggest that this peptide may be acting via an alternative mechanism, either in conjunction with or in addition to, the mechanism by which FDPs have their stimulatory effect. If WTM 35 was acting solely via the same mechanism as that of the FDPs, the increase in stimulatory activity observed at the higher concentrations of WTM 35 would not be expected.

ii) Peptide WTM 36 Stimulates Cell Proliferation

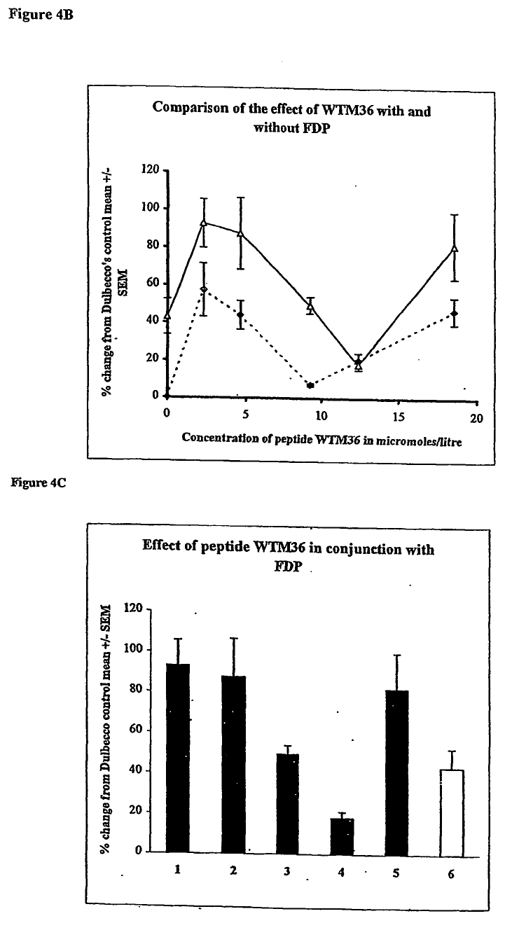

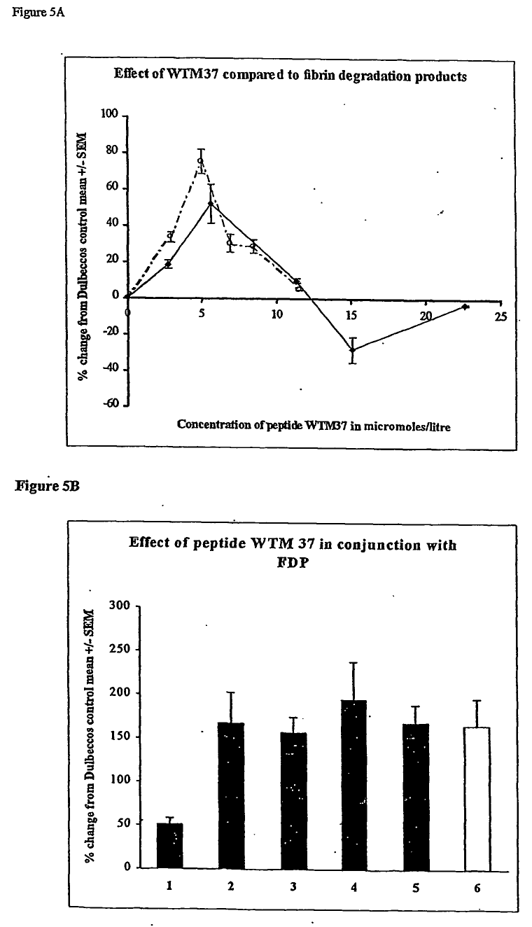

The peptide was tested for modulation of cell stimulation using the same assays as described for WTM35. WTM36 demonstrated a similar pattern of stimulatory activity as that seen for WTM35 (FIG. 4A). At some concentrations tested, the stimulatory effect appears to be additive with the effect produced by FDPs (FIG. 4B). As shown in FIG. 4C, at most concentrations tested, the stimulatory effect seen with 5 μM FDPs is enhanced in the presence of WTM36, although there does appear to be some inhibitory effect at 12.348 μM, with the stimulatory effect recovering at the highest concentration tested (18.53 μM).

Therefore, as with WTM 35, the results obtained with WTM3G at the upper ranges of concentrations tested suggest that WTM36 may also be acting via an alternative mechanism, either in conjunction with or in addition to, the mechanism by which FDPs have their stimulatory effect.

The results obtained with WTM35 and WTM36 suggest that these peptides and their variants may be useful in treatments where sustained growth is required without interference with the effects of fibrin per se. These peptides may therefore be useful in wound healing and related treatments, e.g. treatment of ulcers.

iii) Peptide WTM37 has Low Stimulatory Activity

Peptide WTM37 was tested using the same assays as described for WTM35 and WTM36. In contrast to the results obtained with WTM35 and WTM36, WTM37 shows little inherent stimulatory effect on cell proliferation (FIG. 5A). Moreover, at 15 μmoles, it appears to have an inhibitory effect. There is no increase in stimulatory activity at the upper range of concentrations. As shown in FIG. 5B, the presence of WTM 37 does not enhance FDP-induced stimulation of cell proliferation over the normal range of FDP activity.

Indeed, at low concentrations it may even inhibit the stimulatory effect. (FIG. 5B).

iv) Peptide WTM 33 Inhibits and Stimulates Cell Proliferation

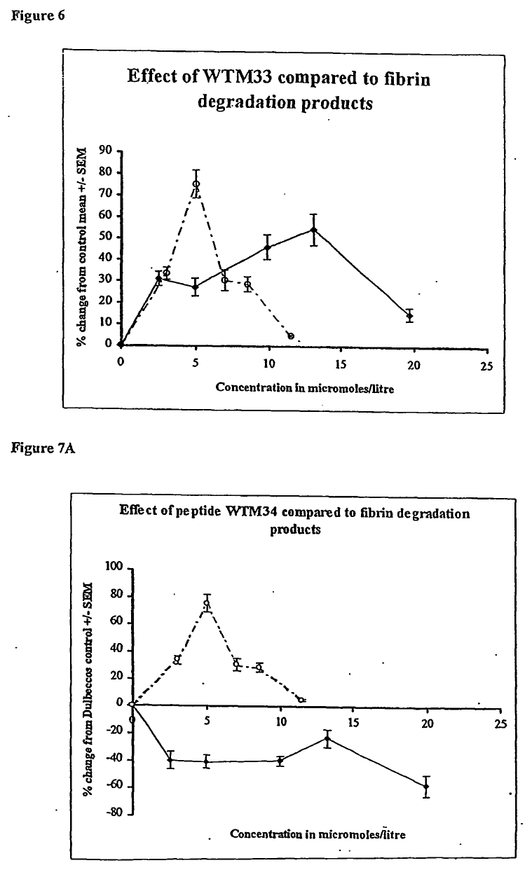

The peptide was tested for modulation of cell stimulation using the same assays as described for the peptides above. At least at very low concentrations, WTM33 shows stimulatory activity over concentrations for which FDPs show no activity (FIG. 6). Therefore, it appears that WTM33 may, like WTM35 and WTM36 be acting via an alternative mechanism, either in conjunction with or in addition to, the mechanism by which FDPs have their stimulatory effect. At higher concentrations WTM33 was inhibitory.

v) Peptide WTM 34 Inhibits Cell Proliferation

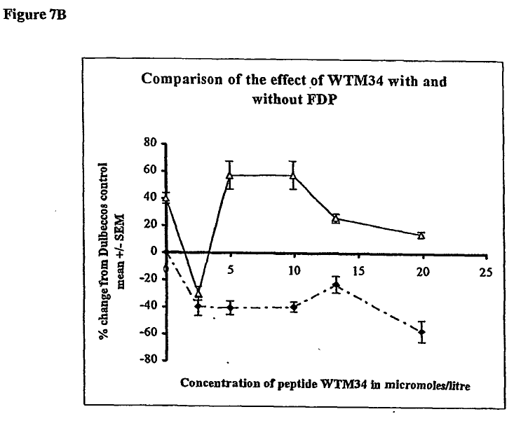

The peptide was tested for modulation of cell stimulation using the same assays as described for the peptides above. In contrast to the results obtained with the other peptides, WTM34 appears to have an inherent inhibitory effect on cell stimulation at all concentrations tested (FIG. 7A). On repeating the assay with the same concentrations of WTM34 in the presence of 5 μM FDPs, the presence of WTM34 at concentrations of 4.633 μM and 9.265 μM does not appear to affect the FDP-induced stimulation of cell proliferation. However, there appears to be significant inhibition of the FDP induced stimulation at lower concentrations, and, possibly at higher concentrations of WTM34. (FIG. 7B).

Knowledge of the location of the active site, even though limited to a few short segments of the protein molecule, allows further exploration of non-immunogenic adjacent areas, within the known molecular structure of fibrin fragment E, which may influence biological activity. Synthetic peptides and analogs of such regions are likely to provide further experimental agents that are potential drugs.

### Example 6

Vessel Counting Assay Using CAM Model

The CAM were prepared as for the tritiated thymidine assay.

Dulbecco's phosphate buffered saline was used as a control. A range of concentrations of peptides WTM33, WTM34, and WTM35 (1.875, 3.75, 7.5, 15, 30, and 60 μg/ml) were added to the CAM. Inoculations of 0.2 ml of the peptide were carried out on days 10,11 and 12 of CAM development, and then fixed using formalin on day 15.

After fixing in formalin for 2 days the CAM were excised and spoke like sections cut from the centre of the CAM to the outer edge. These were embedded in wax and processed on to slides with H&E staining to visualise the vessels in the section. The numbers of vessels per field of vision, over 10 fields were counted using an Olympus microscope. [Eyepieces set at 58, using ×40 (0.65) lens, from the graticule diameter of field is 0.45 mm, and therefore 1 mm is equal to 2.22 fields of view.] The average number of vessels/field of view×2.22 gives number of vessels/mm of CAM.

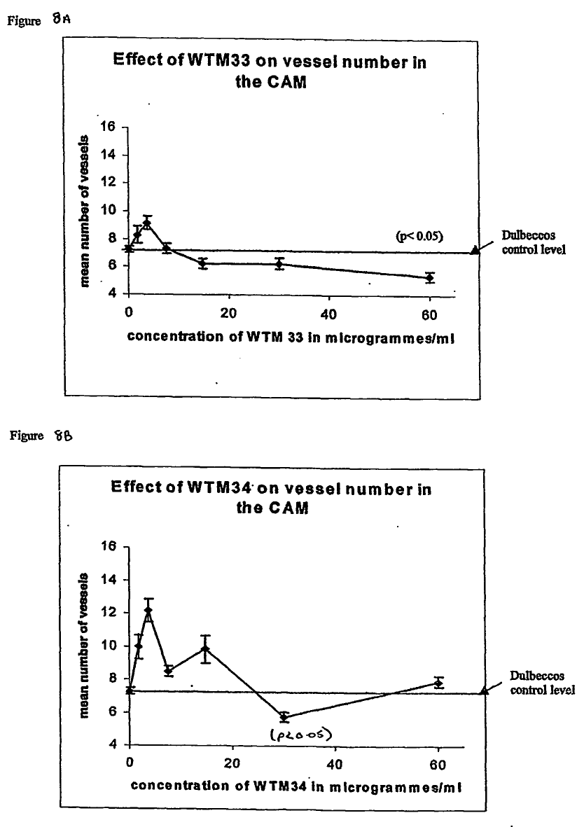

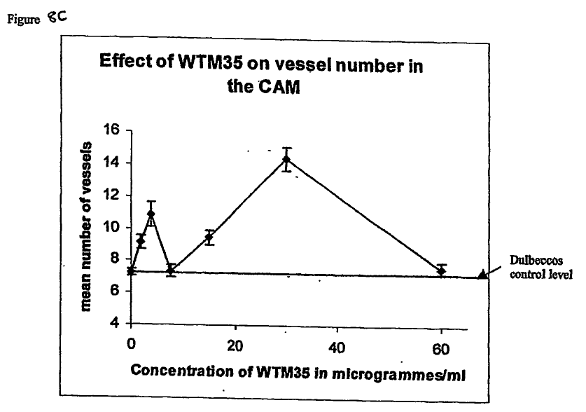

The results showed that Peptides WTM33, WTM34 and WTM35 are capable of modulating the vessel number within the CAM As can be seen in FIGS. 8A, B and C, the three peptides all exhibit modulation of the actual vessel count in the CAM. WTM 33 shows the most potent inhibitory effect which, is in line with its activity in the Angiosys™ system (Example 7). WTM 34 exhibits a more dramatic biphasic reaction but still inhibits significant reduction in vessel formation in the higher dose range. The results for WTM35 show an increase in vessel number, which is consistent with the increase in cell proliferation observed in the CAM proliferation assay.

### Example 7

(i) Use of an In Vitro Model of Angiogenesis.

The recently developed Angiosys™ co-culture angiogenesis assay (TCS Cell Works, Botolph Claydon, UK) was employed to examine the biological activity of the fibrin E analogue peptides WTM33, WTM34, WTM35, WTM36, and WTM37. This assay involves the co-culturing of human umbilical vein endothelial cells (HUVEC) with primary human diploid fibroblasts. In control cultures treated by the standard method as supplied by the manufacturers, both cell populations increase in number to form a confluent fibroblast monolayer on top off which the HUVEC cells form into corded structures. These structures thicken and anastomose by day 14 to form an intricate network of microvessel-like structures that resemble a capillary bed. It has previously been shown that the control conditions in this assay result in an intermediate degree of formation of these microtubules that can be enhanced by angiogenic effectors such as vascular endothelial growth factor (VEGF), or reduced by anti-angiogenic agents such as suramin (33).

The Angiosys™ assay was used as directed by the manufacturer. Briefly, HUVEC and human diploid fibroblasts were admixed, seeded into 24-well culture plates and incubated for 14 days in endothelial cell culture medium (ECM, TCS Cell Works). This medium, which was replaced with fresh every second day, contains angiogenic factors such as VEGF. Fibrin E analogue peptides were dissolved in this same medium to the indicated concentrations and applied to the cells in the same way as control medium. After the incubation period of 14 days, medium was removed and the cells were fixed in situ at room temperature in 70% ethanol and viewed with an MRC 600 confocal microscope (Bio-Rad). Images were captured using a JVC videocamera mounted on the microscope and coupled to a Neotech Image Grabber/PC.

ii) Cytotoxicity Assays

In order to determine whether the fibrin E analogue peptides WTM33, WTM34, WTM35, WTM36, and WTM37 are cytotoxic when applied to the HUVEC and fibroblasts used in the Angiosys™ angiogenesis assay, these reagents were examined in two different cytotoxicity assays. To simplify performance of these assays and discriminate between fibroblasts and HUVEC, cells were grown in monocultures rather than together and then treated with the experimental reagents as described below.

A Neutral Red cell metabolism assay was used to determine the cytotoxicity to HUVEC and fibroblasts of the fibrin E analogue peptides. This assay works on the basis of colorimetric measurement of a red dye which, is reported to be taken up into the lysosomes of uninjured cells. Thus the stronger the colour signal obtained from a well of cells, the greater the percentage of cells remaining viable within that well. HUVEC cells were inoculated into wells of 96 wells at a density of 5×10³ cells per well, in endothelial cell culture medium. Fibroblasts were similarly seeded 5×10³ cells per well. The cells were incubated for 24 hours under standard conditions to permit adherence and growth, and then treated for 72 hours with fresh medium containing fibrin E peptides over the same range of concentrations used in the Angiosys™ angiogenesis assay. For each concentration of each of the six peptides, four replicate wells of each cell type were treated and then assayed. Following incubation with the test compounds, the medium was removed and replaced with 200 μl per well of fresh medium containing 3.57 mg neutral red per 100 ml. The cells were incubated with this neutral red solution for 3 hours, after which this solution was decanted and replaced with 100 μl 1% formaldehyde/1% CaCl₂. This was rapidly decanted and replaced in turn with 200 μl 50% ethanol/1% acetic acid, which was incubated at room temperature for 30 minutes to extract the neutral red from the cells. The plates were then read in a 96 well plate-format spectrophotometer, measuring absorbance at 540 nm. Mean absorbance values from each group of four wells were calculated and expressed as a percentage±standard deviation of the mean value obtained from control wells incubated in the absence of fibrin E analogue peptide.

Cytotoxicity of the fibrin E peptide analogues was also determined by examining cell proliferation, using a commercial BrdU ELISA (Roche Molecular Biochemicals) performed according to the manufacturer's instructions. This assay quantifies DNA synthesis as a measure of cellular proliferation, as stimulation or inhibition of cell replication will require a concomitant change in the rate of DNA synthesis. Measurement of DNA synthesis is achieved by incubating proliferating cells in medium in which thymidine has been replaced with the pyrimidine analogue 5-bromo-2′-deoxyuridine (BrdU). Once incorporated into the cellular DNA, BrdU is measured by immunoassay. Briefly, HUVEC and fibroblasts were seeded as monocultures into 96 well plates at a density of 5×10³ cells per well, to a final volume of 100 μl per well in endothelial cell culture medium. At the end of an initial culture period of 72 hours under standard conditions, while the cells were still proliferating, fibrin E analogue peptides were added to a range of final concentrations, as shown in the FIG. 8. Each treatment concentration of each peptide was administered to four replicate wells. Control cells received standard medium without test compounds. The cells were incubated for 24 hours with the test compounds, before 10 μl of stock BrdU solution (100 μM) was added to give a final concentration of 10 μM BrdU, and the cells were incubated for a further 2 hours. The test medium was then removed from the cells and replaced with 200 μl per well of Fixing/Denaturing solution, which was incubated on the cells for a further 30 minutes at room temperature. This was then replaced with 100 μl per well anti-BrdU-POD (mouse monoclonal clone BMG 6H8, Fab fragments conjugated to peroxidase) prepared as directed, which was left on the cells for a further 90 minutes at room temperature. The antibody conjugate was then removed from the wells by rinsing three times in 200 μl per well of washing buffer (PBS) before 100 μl well substrate solution (tetramethyl-benzidine) was added and incubated at room temperature until colour development was sufficient for photometric detection (approximately 15 minutes). Absorbance at 370 nm was measured on a multi-well plate ELISA plate reader (Dynatech), with a reference wavelength of 492 nm. Mean absorbance values from each group of four wells were calculated and expressed as a percentage±standard deviation of the mean value obtained from control wells incubated in the absence of fibrin E analogue peptide.

(iii) Results

Results are shown in the table below. The table illustrates the stimulator effects of peptide WTM 35 and 36 (0.19-0.75 μg/ml) on HUVEC tubule formation in the Angiosys™ co-culture angiogenesis assay, compared with an untreated control. The table show the average tubule length obtained using an image analysis package for the PC which transforms the visual data into pixels:

Effect of peptides on HUVEC tubule formation
in Angiosys ™ assay

| | | | | | p value |
|---|---|---|---|---|---|
| | | | Mean Tubule | | (students |
| | | | Length (pixels) | S.D. | T-test) |
| WTM33 | (0.75 | μg/ml) | complete inhibition - no tubule | | |
| | (0.38 | μg/ml) | formation | | |
| | (0.19 | μg/ml) | | | |
| WTM34 | (0.75 | μg/ml) | complete inhibition - no tubule | | |
| | (0.38 | μg/ml) | formation | | |
| | (0.19 | μg/ml) | | | |
| WTM35 | (0 | μg/ml) | 9296 | 754 | |
| | (0.75 | μg/ml) | 10879 | 1114 | <0.001 |
| | (0.38 | μg/ml) | 10929 | 986 | <0.001 |
| | (0.19 | μg/ml) | 11330 | 1163 | <0.001 |
| WTM36 | (0 | μg/ml) | 9465 | 1040 | |
| | (0.75 | μg/ml) | 9990 | 1220 | <0.05 |
| | (0.38 | μg/ml) | 1010 | 806 | <0.05 |
| | (0.19 | μg/ml) | 11253 | 1040 | <0.005 |

In summary, as described below, Fibrin E analogue peptides WTM33, WTM34 inhibit HUVEC tubule formation in an in vitro angiogenesis model, but are not cytotoxic. By contrast, at these concentrations, WTM35 and WTM36 stimulate HUVEC tubule formation, and are likewise not cytotoxic.

The five fragment E analogue peptides listed above were tested for their ability to modulate HUVEC tubule formation as described above. To determine whether any observed effect on tubule formation was due to cytotoxicity, the peptides were also examined in two in vitro cytotoxicity assays as described above.

Digital images were taken from single wells of co-cultured HUVEC 15 and fibroblasts treated with 0.18, 3, 6, 12 and 100 μg/ml WTM33, alongside a field from a well of control cells incubated in the absence of peptide. The control showed formation by HUVEC of the network of anastomosing microvessels and served as a benchmark level of tubule formation in the assay. Tubule formation was clearly greatly reduced by WTM33 at all experimental concentrations. WTM33 had no effect on survival of either cell type in the BrdU or Neutral Red assays at any of the test concentrations (3, 12, 100 μg/ml) which span the concentration range, used in the angiogenesis assay. This suggests that the peptide specifically inhibits HUVEC tubule formation via an unknown mechanism, rather than simply by reducing cell numbers as a result of toxicity.

Peptide WTM34 caused the inhibition of HUVEC tubule formation at all concentrations, and had no toxicity in either the BrdU or Neutral Red assays.

The modulation of HUVEC tubule formation by peptide WTM36 may be a biphasic reaction as seen with fibrin degradation products. However even the lowest experimental dose of 0.18 μg/ml showed stimulation of vessels' growth. WTM36 exhibited an effect on the cell proliferation of the cells in the BrdU assay. However, there was no reduction in the viability of the cells, as judged by the Neutral red assay. WTM35, which was also stimulatory, had no toxicity in either the BrdU or Neutral Red assays.

At a concentration of 12 μg/ml peptide WTM37 exhibited a degree of inhibition of HUVEC tubule formation. The magnitude of this effect was increased at higher doses, and WTM37 showed no toxicity to either cell type in either assay of cytotoxicity.

Thus peptides WTM33 and WTM34 were the most inhibitory of the fibrin E analogues in the Angiosys™ anti-angiogenic assay. At the lowest applied doses of 3 μg/ml both these reagents markedly reduced the formation of tubules by the HUVEC cells, and this effect was not enhanced at higher concentrations as determined by eye. The other fibrin E peptide analogues also inhibited formation of tubules by HUVEC.

WTM 35 and 36 also exhibited an ability to stimulate vessel growth at lower concentrations (0.18 μg/ml), making these of potential use for a therapeutic agent in poorly healing wounds, such as diabetic leg ulcers. The results of two different cytotoxicity assays indicated that none of the fibrin E analogue peptides had a significant effect on cell survival at any concentration examined, which overlapped with the concentrations at which these reagents were demonstrated to inhibit tubule formation in the Angiosys™ assay. However, WTM36 displayed a potential for reduction in cell proliferation in the BrdU assay at higher concentrations.

Results produced with the Angiosys™ co-culture angiogenesis system indicate that the five fibrin E peptides examined are all capable of modulating, to varying degrees, the formation of microvessel-like tubules by primary human endothelial cells. Two assays of cytotoxicity have further shown that the peptides are non-toxic to the two cell types used in the Angiosys™ system, which suggests the peptides may specifically inhibit some aspect of tubule formation, by an unknown mechanism. As seen in FIG. 8A peptide WTM33 can inhibit vessel formation in the CAM at relatively high doses. Also FIG. 7A and in FIG. 8B, peptide WTM34 inhibits cell proliferation and vessel formation in the CAM respectively, and so it is easy to reconcile the results found using this peptide in the Angiosys™ system with findings in the CAM.

It should be noted that the concentrations used in the tubule formation experiments are not directly comparable with those used in the CAM assay. For the first experiments with the Angiosys™ system, the peptides were applied in relatively high concentration compared with the range used in the CAM assay. Also the effective concentration may be much higher in the culture assay than in the CAM assay, since agents are removed from the area by the chick circulatory system. This means that results observed in the CAM are due to a short pulse of agent and therefore show the immediate effect. In the Angiosys™ system the peptide is refreshed every two days and is in constant contact with the cells as there is no system for removal.

### Example 9

Peptides in Two Animal Models Used

Pulmonary Tumour Metastasis Model

In the metastasis model, Lewis Lung Carcinoma (LLC) cells are injected into the tail of the mouse, lodge in the lungs, and grow and spread rapidly.

In more detail, groups of five C57BL/6J mice were injected with 5×10⁴ LLC cells via the tail vein and were subsequently treated intraperitoneally with various concentrations of tested peptides or equal volume of diluent control. The treatment was initiated three days after tumor cell inoculation. Two weeks after the intravenous injection of tumor cells, the mice were sacrificed and necropsied. The lungs were removed, weighed and compared to untreated controls, and to normal lungs without tumors. Results were analyzed for statistical significance using the Student's t-test.

Primary Tumour Model

In the primary tumour model, LLC cells are injected under the skin of the mouse and the resulting tumour mass is measured.

In more detail, groups of five C57BL/6J mice were injected subcutaneously with 2.5×10⁵ Lewis lung carcinoma cells and were subsequently treated with daily i.p. doses of the peptides, or equal volume of diluent control. The treatment was initiated a week after tumor cell inoculation, at which point tumors have a volume of approximately 100 mm³, and continued every day for 10 to 12 days. Tumor growth was recorded every other day using calipers to measure the tumor dimensions. Tumor volume was calculated using the following formula: (length)ₓ(width)₂×(p/6). The tumor size in the treated animals was compared to the tumor size in the control animals. Statistical analysis was performed using the Student's t-test.

Results

Purely visually, mice treated with the peptides appeared normal, with no obvious toxicity other than that attributable to the developing tumours.

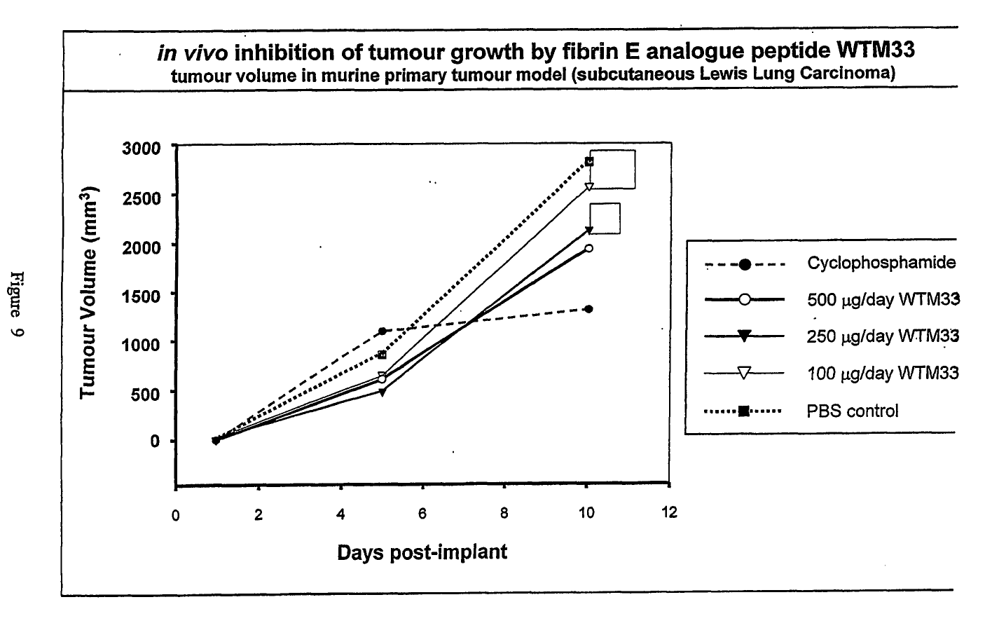

FIG. 9 shows the performance of various concentrations of WTM33 in primary tumour model, as compared with a cyclophosphamide. The peptide exhibited a >30% decrease in the size of the tumour at 500 microgrammes/day, and it is believed that further optimisation of the dose may improve this.

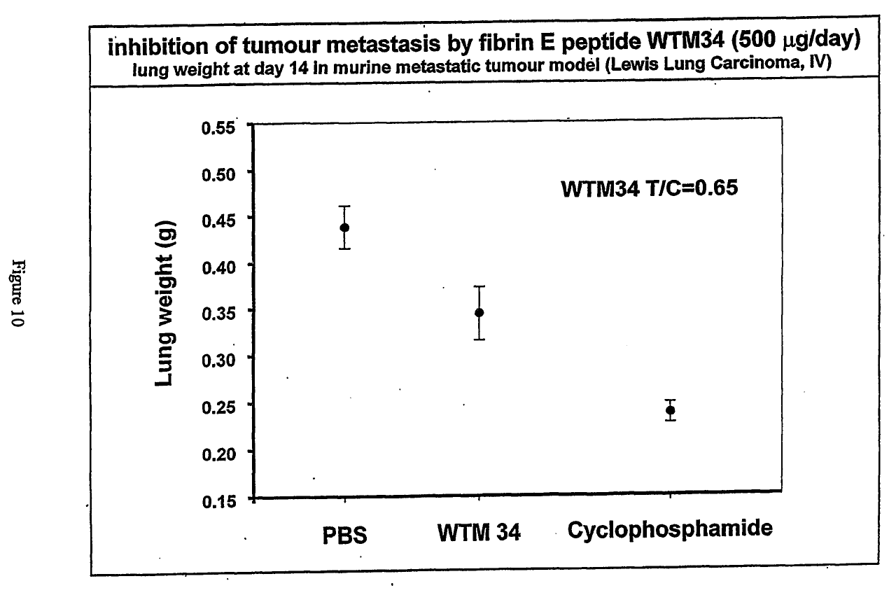

FIG. 10 compares the performance of WTM34 in metastatic tumour model with +ve and −ve controls. This peptide gave an approximately 35% reduction in the increase in weight of the lungs.

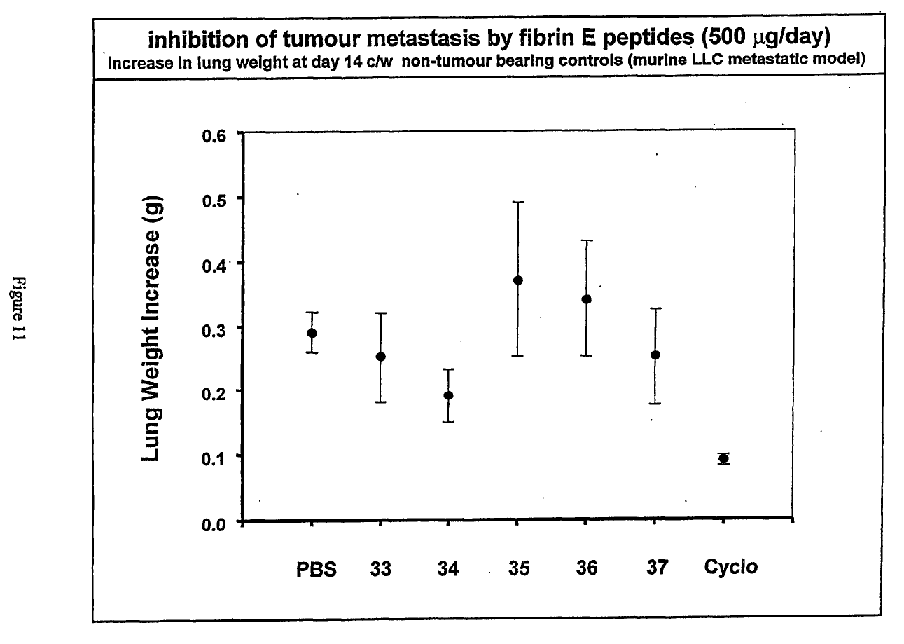

FIG. 11 shows effect of five fibrin E peptide analogues on weight of lungs of mice injected with LLC in the metastatic tumour model (increased lung weight being due to metastatic tumour masses). As can be seen WTM34 in particular gave striking results as shown in FIG. 12. WTM35 and 36 gave results consistent with a pro-angiogenic \ cell proliferation role.

### REFERENCES

1 McKenzie R, Pepper D S, Kay A B. The generation of chemotactic activity for human leucocytes by the action of plasmin on human fibrinogen. Thromb Res 1975; 6: 1-8.

2 Thompson WD, Campbell R, Evans AT. Fibrin degradation and angiogenesis: quantitative analysis of the angiogenic response in the chick chorioallantoic membrane. J Pathol 1985; 145: 27-37.

3 Thompson W D, Evans A T, Campbell R. The control of fibrogenesis: stimulation and suppression of collagen synthesis in the chick chorioallantoic membrane with fibrin degradation products, wound extracts and proteases. J Pathol 1986; 148: 207-215.

4 Thompson W D, Smith E B, Stirk C M, Marshall F I, Stout A J, Kocchar A. Angiogenic activity of fibrin degradation products is located in fibrin fragment E. J Pathol 1992; 168: 47-53.

5 Romer J., et al. Impaired wound healing in mice with a disrupted plasminogen gene. Nature Med. 2, 287-292 (1996).

6 Bugge T H, Kombrinck K W, Flick M J, Daugherty C C, Danton M J, Degen J L. Loss of fibrinogen rescues mice from the pleiotropic effects of plasminogen deficiency. Cell 1996; 87: 709-19.

7 Thompson W D, Harvey J A, Kazmi M A, Stout A J. Fibrinolysis and angiogenesis in wound healing. J Pathol 1991; 165: 311-318.

8 Thompson W D, McNally S J, Ganesalingam N, McCallion D S E, Stirk C M, Melvin W T. Wound healing, fibrin and angiogenesis. In: Molecular, Cellular and Clinical Aspects of Angiogenesis. Ed M Maragoudakis, Plenum Press, New York, 1996. pp 161-172

9 Sarembock I J, Gertz S D, Gimple L W, Owen R M, Powers E R, Roberts W C. Effectiveness of recombinant desulphatohirudin in reducing restenosis after balloon angioplasty of atherosclerotic femoral arteries in rabbits. Circulation 1991; 84, 232-243

10 Schwartz S M. Serum-derived growth factor is thrombin? J Clin Invest 1993; 91: 4

11 Darrow A L, Fung-Leung W P, Ye R D, Santulli R J, Cheung W M, Derian C K, Burns C L, Damiano B P, Zhou L, Keenan C M, Peterson P A, Andrade-Gordon P R. Biological consequences of thrombin receptor deficiency in mice. Thromb Haemost 1996; 76: 860-866.

12 Herbert J M, Guy A F, Lamarche I, Mares A M, Savi P, Dol F. Intimal hyperplasia following vascular injury is not inhibited by an antisense thrombin receptor oligodeoxynucleotide. J Cell Physiol 1997; 170: 106-114.

13 Naito M, Sabally K, Thompson W D, Stirk C M, Smith E B, Benjamin N. Stimulation of proliferation of smooth muscle cells in culture by fibrin degradation products. Fibrinolysis 1996; 10 (Suppl 4): 1-26.

14 Woodward M, Lowe G D O, Rumley A, Tunstall-Pedo H. Fibrinogen as a risk factor for coronary heart disease and mortality in middle-aged men and women. Eur Heart J 1998; 19: 55-62.

15 Lowe G D O, Yarnell J W G, Sweetnam P M, Rumley A, Thomas H F. Fibrin D-dimer, tissue plasminogen activator, plasminogen activator inhibitor, and the risk of major ischaemic heart disease in the Caerphilly study. Thromb Haemost 1998; 79: 129-133.

16 Lip GYH, Lowe GDO. Fibrin D-dimer: a useful clinical marker of thrombogenesis. Clin Sci 1995; 89: 205-214.

17 Tschopl M, Tsakiris D A, Marbet G A, Labs K-H, Jager K. Role of hemostatic risk factors for restenosis in peripheral arterial occlusive disease after transluminal angioplasty. Arterioscler Thromb Vasc Biol 1997; 17: 3208-3214.

18 Smith E B, Keen G A, Grant A, Stirk C. Fate of fibrinogen in human arterial intima. Atherosclerosis 10, 263-275, 1990.

19 Duguid J B. Thrombosis as a factor in the pathogenesis of coronary atherosclerosis. J Path Bact 1946; 58: 207-212.

20 Thompson W D, McGuigan C J, Snyder C, Keen G A, Smith E B. Mitogenic activity in human atherosclerotic lesions. Atherosclerosis 1987; 66: 85-93

21 Thompson W D, McGuigan C J, Snyder C, Keen G A, Smith E B. Mitogenic activity in human atherosclerotic lesions. Atherosclerosis 1987; 66: 85-93.

22 C M Stirk, A Kochhar, E B Smith, W D Thompson. Presence of growth-stimulating fibrin degradation products containing fragment E in human atherosclerotic plaques. Atherosclerosis 1993; 103: 159-169.

23 Dvorak H F, Nagy J A, Berse B, Brown L F, Yeo K T, Dvorak A M, van de Water L, Sioussat T M, Senger D R. Vascular permeability factor, fibrin, and the pathogenesis of tumor stroma formation. Ann NY Acad Sci 1992; 667: 101-111.

24 Zacharski L R, Schned A R, Sorenson G D. Occurrence of fibrin and tissue factor antigen in human small cell carcinoma of the lung. Cancer Res 1983; 43: 3963-3968.

25 Zacharski L R, Memoli V A, Rousseau S M. Coagulation-cancer interaction in situ in renal cell carcinoma. Blood 1986; 68: 394-399.

26 Rickles F R, Hancock W W, Edwards R L, Zacharski L R. Antimetastatic agents. I. Role of cellular procoagulants in the pathogenesis of fibrin deposition in cancer and the use of anticoagulants and/or antiplatelet drugs in cancer treatment. Semin Thromb Hemost 1988; 14: 88-94.

27 Scott J K, and Smith G P. Searching for peptide ligands with an epitope library. Science 1990; 249: 186-390.

28 Parmley S F, and Smith G P. Antibody-selectable filamentous Fd phage vectors—affinity purification of target genes. Gene 1988; 73: 305-318.

29 Thompson W D, Smith E B, Stirk C M, Wang J. Fibrin degradation products in growth stimulatory extracts of pathological lesions. Blood Coagulation and Fibrinolysis 1993; 4: 113-116.

30 Thompson W D, Brown F I. Measurement of angiogenesis: mode of action of histamine in the chick chorioallantoic membrane is indirect. Int J Microcirc 1987; 6: 343-357.

31 Thompson W D, McNally S J, Ganesalingam N, McCallion DSE, Stirk CM, Melvin WT. Wound healing, fibrin and angiogenesis. In: Molecular, Cellular and Clinical Aspects of Angiogenesis. Ed M Maragoudakis, Plenum Press, New York, 1996. pp 161-172

32 Thompson W D, Stirk C M, Keating A J, Reid A, Smith E B, Melvin W T. Fibrin degradative pathways in healing, atherosclerosis and tumour invasion. In: Angiogenesis: Models, Modulators and Clinical Applications. Ed M Maragoudakis, Plenum Press, New York, 1998 pp 233-240.

33 Bishop E T, Bell G T, Bloor S, Broom I J, Hendry N F K, Wheatley D N. An in vitro model of angiogenesis: basic features. Angiogenesis 1999; 3: 335-344.

1. A peptide consisting of a sequence selected from the group consisting of:
SLRPAPPPISGGGYR (SEQ ID NO:1) (WTM33)
LHADPDLGVLCPTGC (SEQ ID NO:2) (WTM34)
QLQEALLQQERPIRN (SEQ ID NO:3) (WTM35)
SVDELNNNVEAVSQT (SEQ ID NO:4) (WTM36)
SSSSFQYMYLL (SEQ ID NO:5) (WTM37)

2 a peptide variant of a peptide as claimed in claim 1, which variant has variations in one, two, three or four amino acids of the reference peptide, said variant being capable of modulating a fibrin fragment e activity.

3 A peptide variant as claimed in claim 2 wherein the or each variation is a conservative substitution.

4 A peptide according to claim 2 or claim 3 wherein the said activity is modulation of cell proliferation or angiogenesis.

5 A peptide according to claim 4 wherein the modulation is either stimulatory or inhibitory according to dosage.

6 A fusion peptide which comprises a first portion having the amino acid sequence of a peptide defined in any one of the preceding claims and a second portion, attached to the N- or C-terminus of the first portion, which comprises a sequence of amino acids not naturally contiguous to the first portion.

7 A fusion peptide as claimed in claim 6 wherein the second portion comprises a membrane translocation sequence.

8 A method of modulating a fibrin fragment E activity in a cell or tissue comprising bringing the cell or tissue into contact with a peptide according to any one of the preceding claims.

9 A method according to claim 8 wherein the said modulation of activity is stimulation of cell proliferation or angiogenesis.

10 A method according to claim 8 wherein the said modulation of activity is inhibition of cell proliferation or angiogenesis.

11 A method according to claim 10 wherein the cell proliferation is induced by a fibrin degradation product

12 A peptide according to any one of claims 1 to 7 for use in a method of treatment of the human or animal body.

13 A peptide according to claim 12 for use in a method of treatment of a disease or disorder which responds to stimulation of cell proliferation or angiogenesis.

14 A peptide according to claim 13 wherein the treatment is for:
wound healing, increasing effectiveness of skin graphs, revascularisation of heart muscle or limb replacement surgery.

15 A peptide according to claim 13 or claim 14 wherein the disease is ischaemia.

16 A method according to claim 9 or a peptide according to any one of claims 13 to 15 wherein the peptide is either:
QLQEALLQQERPIRN (SEQ ID NO:3) (WTM35), or
SVDELNNNVEAVSQT (SEQ ID NO:4) (WTM36)
or a variant peptide of either as claimed in claim 2.

17 A peptide according to claim 12 for use in a method of treatment of a disease which responds to inhibition of cell proliferation or angiogenesis.

18 A peptide according to claim 17 wherein the treatment is for: reduction of scarring; prevention of restenosis.

19 A peptide according to claim 13 or claim 14 wherein the disease or disorder is selected from atherogenesis, rheumatoid arthritis, diabetes, renal disease, psoriasis, macular degeneration, cancer.

20 A method according to claim 10 or a peptide according to any one of claims 17 to 19 wherein the peptide is either
SLRPAPPPISGGGYR (SEQ ID NO:1) (WTM33), or
LHADPDLGVLCPTGC (SEQ ID NO:2) (WTM34)
or a variant peptide of either as claimed in claim 2.

21 Use of a peptide according to any one of claims 1 to 7 in the preparation of a medicament for the inhibition or stimulation of cell proliferation or angiogenesis.

22 A composition comprising a peptide according to any one of claims 1 to 7 in association with a pharmaceutically acceptable carrier or diluent.

23 An antibody or fragment thereof capable of selectively binding to a peptide according to claim 1.

24 An antibody according to claim 23 which is a monoclonal antibody or a polyclonal antibody or antiserum.

25 Use of an antibody or binding fragment according to claim 23 or claim 24 in a method of labelling a fibrin fragment E binding partner.

26 Use as claimed in claim 25 wherein the binding partner is a receptor.

27 A method of identifying a compound capable of modulating a fibrin fragment E activity, wherein said method comprises the steps of:
providing an antibody or binding fragment according to claim 23 or claim 24;
contacting said antibody or binding fragment with a putative modulator compound; and
determining whether said antibody or binding fragment is able to selectively bind to the compound.

28 A method according to claim 27 wherein the compound is provided in the form of an expression or chemical library.

29 A method according to claim 27 or claim 28 further comprising the step of testing the ability of the modulator to modulate cell proliferation and/or angiogenesis.

30 A process for producing a modulator comprising the step of identifying the modulator according to the method of any one of claims 27 to 29.

31 An isolated nucleic acid molecule encoding a peptide according to any one of claims 1 to 7, said nucleic acid not being naturally contiguous with nucleic acid sequence encoding native fibrin.

32 An expression vector comprising an isolated nucleic acid as defined in claim 31 operably linked to a promoter.

33 A host cell carrying a vector according to claim 32.
